(19) United States
(12) Patent Application Publication
(10) Pub. No.: US 2025/0107876 A1
(43) Pub. Date: Apr. 3, 2025

(54) MAGNETIC POSITIONING SYSTEMS AND METHODS FOR DENTAL PROCEDURES AND APPLICATIONS

(71) Applicant: CLOUDNAV INC., San Jose, CA (US)

(72) Inventors: Erik E. ANDERSON, Mountain View, CA (US); Nathan Charles ROYER, San Jose, CA (US); Benjamin KOKES, San Mateo, CA (US); Jeffery DECH, San Jose, CA (US); Jon MAIARA, Malden, MA (US)

(21) Appl. No.: 18/900,740

(22) Filed: Sep. 29, 2024

Related U.S. Application Data
(60) Provisional application No. 63/586,932, filed on Sep. 29, 2023.

(51) Int. Cl. A61C 19/04; A61B 34/20

(52) U.S. Cl. CPC A61C 19/04; A61B 2034/2046

(57) ABSTRACT

Devices, systems and methods for estimating a position and an orientation of a dental tool are described. An example system for estimating a position and an orientation of a dental tool includes multiple electromagnets are removably affixed to an object positioned in a patient's mouth, the object being movable at least based on movements of the patient's mouth, and multiple magnetometers affixed to a dental tool that is non-stationary. The system further includes at least one processor configured to drive the plurality of electromagnets with at least one programmed signal, receive, from the plurality of magnetometers, magnetic field measurements, and determine, based on the magnetic field measurements, the position and the orientation of the dental tool relative to the object.

### CROSS-REFERENCE TO RELATED APPLICATION

This application claims the benefit of priority to U.S. Provisional Patent Application No. 63/586,932, entitled “MAGNETIC POSITIONING SYSTEM FOR DENTAL APPLICATIONS” and filed on Sep. 29, 2023. The entire contents of the before-mentioned patent application are incorporated by reference as part of the disclosure of this application.

### TECHNICAL FIELD

This application generally relates to magnetic positioning, and more particularly, to estimating the position and orientation of a dental tool using magnetometers and beacons.

### BACKGROUND

In dental treatment, dental handpieces come in several shapes and most commonly are either straight or have a swan neck. Handpieces can alternatively be driven by electric or pneumatic drive motors. Handpieces are used to drive tools during various dentistry operations, such as filling cavities, cleaning and the fitting of dental implants. In all these procedures, consistent precision in positioning the dental handpiece is paramount for a successful outcome.

### SUMMARY

Embodiments of the disclosed technology enable, in some examples, estimating the position and/or orientation of a dental handpiece for the purpose of precisely automating the use of such tool (e.g., as part of a robotic system). The described implementations may be used to improve the efficacy of several dental procedures, including preparation for a filling, crown placement, a root canal, and/or dental implant surgery. This can be achieved by the system advantageously aiding a dentist and/or medical provider by providing feedback in one of many forms, including visual, haptic, and the like.

In an example aspect, a system for estimating a position and an orientation of a dental tool includes a plurality of electromagnets removably affixed to an object positioned in a patient's mouth, and a plurality of magnetometers affixed to the dental tool. Herein, the object is movable at least based on movements of the patient's mouth, and the dental tool is non-stationary. The system further includes at least one processor that is configured to drive the plurality of electromagnets with at least one programmed signal, receive, from the plurality of magnetometers, magnetic field measurements, and determine, based on the magnetic field measurements, the position and the orientation of the dental tool relative to the object.

In another aspect, a method for estimating a position and an orientation of a dental tool includes determining a position of each of a plurality of electromagnets removably affixed to an object in a patient's mouth, driving the plurality of electromagnets with at least one programmed signal, receiving, from a plurality of magnetometers removably affixed to the dental tool that is non-stationary, magnetic field measurements when the dental tool is adjacent to the object, and determining, based on the magnetic field measurements, the position or the orientation of the dental tool relative to the object. In this example, the object is movable at least based on movements of the patient's mouth, and the position is determined within a tolerance.

In yet another aspect, a dental system includes a hand-held dental tool configured to accommodate either, but not both, of (a) a plurality of magnetometers or (b) a plurality of electromagnets, and where the other of the plurality of magnetometers or the plurality of electromagnets not accommodated by the hand-held dental tool are configured to be affixed to an object within a patient's mouth. This system further includes one or more processors configured to drive the plurality of electromagnets with at least one programmed signal, receive, from the plurality of magnetometers, magnetic field measurements, and determine, based on the magnetic field measurements, a position and an orientation of the hand-held dental tool relative to the object. Herein, both the plurality of electromagnets and plurality of magnetometers are moveable at least due to movements of the hand-held dental tool and movements of the patient's mouth, respectively.

In yet another example aspect, the above-described method may be implemented by an apparatus or device that includes a processor and/or memory.

In yet another example aspect, this method may be embodied in the form of processor-executable instructions and stored on a computer-readable program medium.

The subject matter described in this patent document can be implemented in specific ways that provide one or more of the following features.

### BRIEF DESCRIPTION OF THE DRAWINGS

FIG. 1 illustrates a flowchart for an example iterative localization method.

FIGS. 2A-2E illustrate various configurations for multiple beacons.

FIG. 3A illustrates an example of an off-axis beacon for sensor measurements.

FIG. 3B illustrates an example of an on-axis beacon for sensor measurements.

FIGS. 4A and 4B illustrate examples of scoring a simplified three-dimensional (3D) model based on a point cloud.

FIGS. 5A and 5B illustrate examples of scoring a more realistic 3D model.

FIG. 6 illustrates an example of ambiguous localization for a linear configuration of magnets (or beacons).

FIGS. 7A and 7B illustrate another example of ambiguous localization relative to a magnetometer board on the dental handpiece.

FIGS. 8A and 8B illustrate yet another example of ambiguous localization for a parallel configuration of magnets (or beacons) with oppositely located poles.

FIGS. 9A and 9B illustrate an example of using a gravity vector to disambiguate the ambiguous localization example shown in FIGS. 12A and 12B.

FIG. 10 illustrates another example of disambiguating the ambiguous localization example shown in FIGS. 8A and 8B.

FIG. 11 illustrates an example of a jointed sleeve that can be used for guided dental surgery, in accordance with the described embodiments.

FIGS. 12A and 12B illustrate a 3D model of an example positioning system in a visualization software and a prototype of the example positioning system, respectively.

FIG. 13 illustrates a 3D model of another example positioning system in the visualization software.

FIG. 14 illustrates an example of the printed circuit board (PCB) assembly.

FIG. 15 illustrates an example of the dental handpiece (or tool).

FIG. 16 highlights pneumatic tubes in the dental handpiece of FIG. 6.

FIG. 17 illustrates a half-section view of a prototype of the dental handpiece.

FIGS. 18A-18C illustrate various magnetometer configurations on the dental handpiece.

FIG. 19 illustrates a flowchart of an example method for estimating a position and an orientation of a dental tool.

FIG. 20 is a block diagram illustrating an example system configured to implement embodiments of the disclosed technology.

### DETAILED DESCRIPTION

Devices, systems, and methods for estimating a position and an orientation of a dental tool (or handpiece) are described. Section headings are used in the present document to improve readability of the description, and do not in any way limit the discussion or the embodiments (and/or implementations) to the respective sections only.

### 1 Introduction

Dental implant surgery is a procedure that replaces tooth roots with metal, screwlike posts and replaces damaged or missing teeth with artificial teeth that look and function much like real ones, and requires precise implant placement. Traditional methods of achieving the precision needed include using a template to help guide the dentist in the drilling and placement of the implant. Advancement in imaging technology has allowed for precise mapping and localization of a dental drill relative to a patient. There are many reasons why robot assisted, or automated, placement can improve the success of such a procedure (e.g., Section 3.1 of Dental Robotics: A Disruptive Technology by Ahmad et al. in “Sensors (Basel)” 2021 May; 21(10): 3308). However, the precision required for such a procedure to be successful is high, with variations in anatomy making this procedure tricky. For example, the YOMI® robot (developed by Neocis and retailing at ˜$150,000), uses a video image-based system to determine the location of the dental drill relative to the teeth, advertising 1 mm level accuracy.

Furthermore, current video-based localization systems typically lose the ability to accurately track position as soon as drilling starts due to debris such as blood, saliva, and bone and tooth dust. The described magnet-based localization implementations are not susceptible to any visual impairments that result as part of the dental procedure.

For other procedures, it is desirable to leave as much of the healthy part of the tooth as possible. Precision drilling for fillings, crowns, and root canals can also greatly decrease the amount of healthy tooth removed. There are currently no magnet-guided systems on the market, and the disclosed embodiments advantageously provide better than 0.3 mm level accuracy, which can help robot-assisted dental surgery and procedures.

In addition, the described implementations can work in the presence of other alternating current (AC) or direct current (DC) magnetic fields, which is particularly beneficial in dental applications because some dental implants make use of strong permanent magnets to affix the tooth to the anchor. Additionally, the system can be deployed with a robot that has motors generating a magnetic field, in the presence of electrical systems (e.g., both US power at 60 Hz and international frequencies).

As used in this document, a beacon is an object that generates a known magnetic field pattern. In an example, the beacon is an alternating current (AC) magnet (and referred to simply as a magnet). In another example, the beacon is an electromagnet (e.g., a wire wound into a coil, with the wire turns wound around a magnetic core). In yet another example, the beacon is a permanent magnet.

As used in this document, an object includes a collection of smaller objects that are rigidly connected to each other, and hence when the object is moved, will experience the same displacement and orientation change.

As used in this document, a beacons object is an object in the localization system comprising enough beacons to uniquely determine its position and orientation. The set of beacons exhibiting the same pattern necessarily comprise a single object as the pattern from all the beacons will be additive. In an example, a well-defined model of this object, which is needed to determine its location, can be determined based on the set of beacons operating at the same frequency and/or generating the same pattern. In another example, the beacons are rigidly connected to teeth, dentures, or the like.

As used in this document, a sensors object is an object including multiple magnetic field sensors, such as MEMS magnetometers, that are configured to collect magnetic field measurements. In an example, the multiple magnetometers are rigidly connected to a circuit board with a memory and a processor, a dental handpiece, or a registration probe.

Embodiments of the disclosed technology, in a first operation, include placing and rigidly attaching multiple beacons to teeth or another object, then using multiple sensors (e.g., magnetometers) to collect measurements, and finally employing a localization method to determine the relative position and/or orientation of each of the sensors relative to the each of the multiple beacons.

In a second operation (that relies on the first operation being completed), a registration system is used to determine (a) the relative position of the beacons to the teeth or the other object and (b) the relative position of the sensors to a dental handpiece or other object rigidly connected to the sensors. Then, the relative positions and registration positions are combined to determine the relative position of the dental tool or other object to the teeth or the other object.

In a third operation (which also relies on the first operation being completed), a calibration system compensates for imperfections in size, orientation, location, pattern, and strength of the beacons.

### 2 Examples of Localization

As part of the first operation discussed in Section 1, magnetism-based localization solutions, which typically use magnetic sensors (e.g., magnetometers) to sense a magnetic field generated by anchors in fixed and known positions, and then try to estimate the relative positions of the anchors, are employed. Magnetism-based localization can be configured to use various technologies, e.g., the Hall effect, giant magnetoresistance (GMR), tunneling magnetoresistance (TMR), and the like.

2.1 Single beacon localization

A magnetometer sensor will measure the magnetic field vector, sometimes referred to as magnetic flux density, at its location. Each set of magnets (or beacons) will contribute to the value of this measurement. Each magnetic dipole source will add a contribution at a position \vec{r} from the sensor that obeys the equation:

$$B ( \vec{r} ) = \frac{{μ}_{0}}{4 π} \frac{3 \hat{r} ( \vec{m} \cdot \hat{r} ) - \vec{m}}{{r}^{3}} .$$

The location of the sensor \vec{r} can be found using the inverse of the above equation.

In some embodiments, a total of five measurements are required if the beacon strength is known, and at least six measurements if it is not. The location of the sensor and the dipole strength vector can be described by the parameter set (x, y, z, θ, ϕ, m) where x, y, and z describe the location of the sensor relative to the dipole in Cartesian co-ordinates, θ and ϕ describe the angular orientation of the dipole relative to the sensor and m is the dipole strength. As the dipole strength m is already known, there are five remaining unknown parameters to be solved. In embodiments where a three-axis magnetic sensor is used, the five measurements require the use of two sensors, since three measurements, in x, y, and z, are taken by the sensor for each beacon.

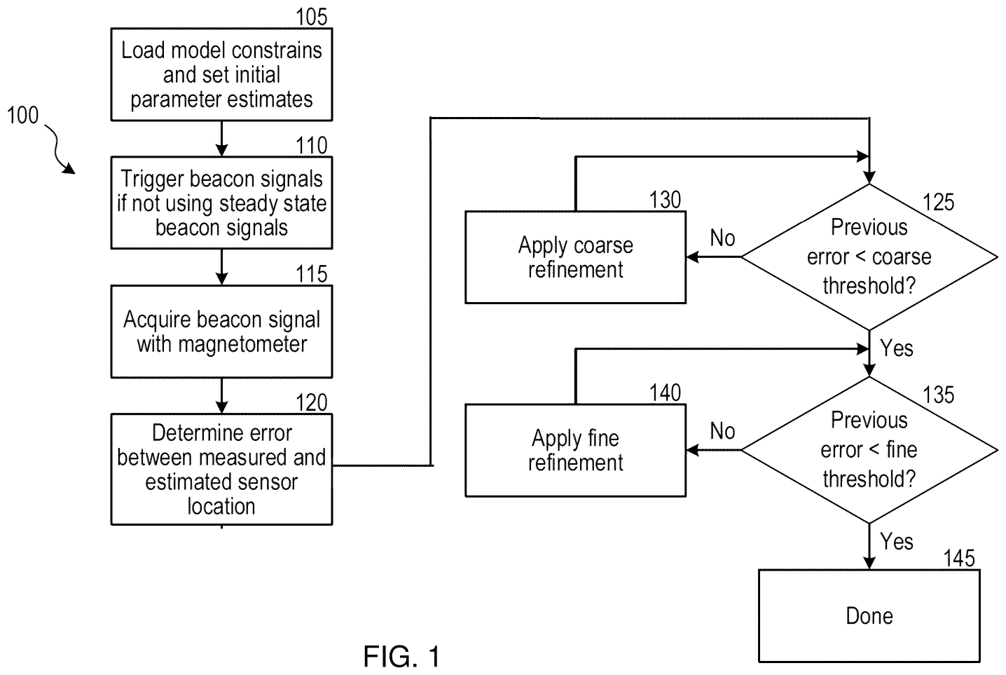

In some embodiments, an iterative method can be used to solve the inverse problem, as illustrated in FIG. 1. As shown therein, the method starts by loading known constraints on the solution and an initial guess (105). The reference beacons are triggered to emit a magnetic signal, if not already operating in a steady state mode (110). The magnetic sensors then acquire signals from each reference beacon (115) either simultaneously or in serial, as required by the specific embodiment, and apply any required signal processing to determine the magnetic field strength of the signal from each beacon. At each iteration, the error between the measured magnetic field and the expected strength using the sensor's presumed location is computed (120). This error is used as the convergence metric for the solution single-beacon localization problem.

Herein, the gradient of the error is used to update the parameter values. The derivative with respect to each parameter is calculated. The parameters are updated by a product of the negative gradient and a proportionality constant (e.g., the learning rate). Some embodiments can separate the search into a coarse and fine grain method, as shown in FIG. 1. The difference between the two refinement methods is the magnitude of the learning during each iteration. The coarse stage will use a larger learning rate than the fine stage. If the error is below a coarse grain threshold (125), the coarse update is skipped (“Yes” at 125); otherwise (“No” at 125), the coarse refinement is applied (130). Similarly, iterations at the fine update size continue (135, 140) until a second, smaller fine grain threshold is reached at which point, the iterations stop (145).

It is noted that the localization solution will only provide the relative position and direction of the beacon to the magnetometers. If the magnetometers are fixed in space, the absolute position of the beacon can be found as the beacon is symmetric about the dipole axis. If the beacon is fixed in space, the location of the sensors is ambiguous and can be one of many positions rotated about the dipole axis (and which is discussed in Section 5).

### 2.2 Multiple Beacon Localization

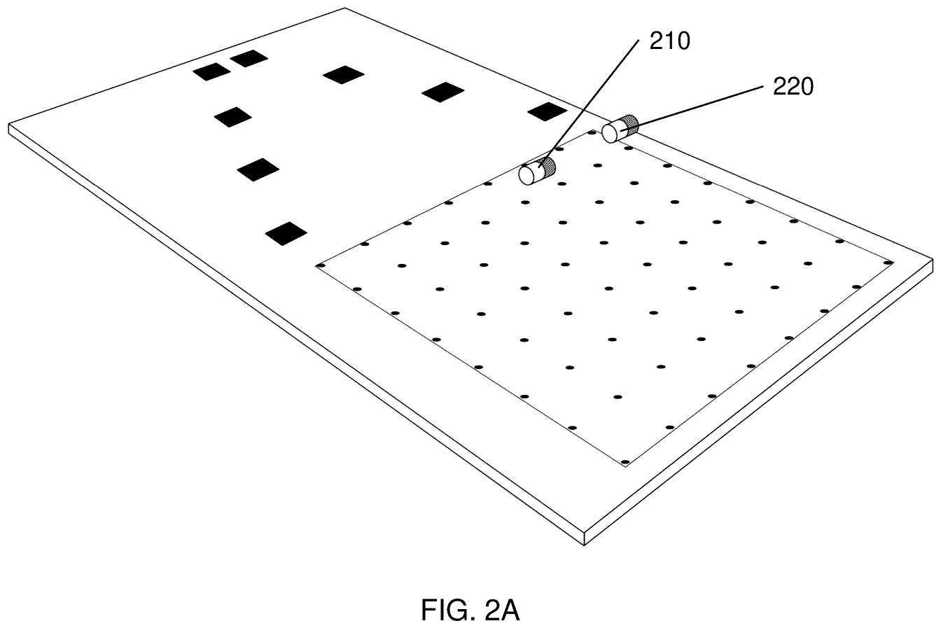

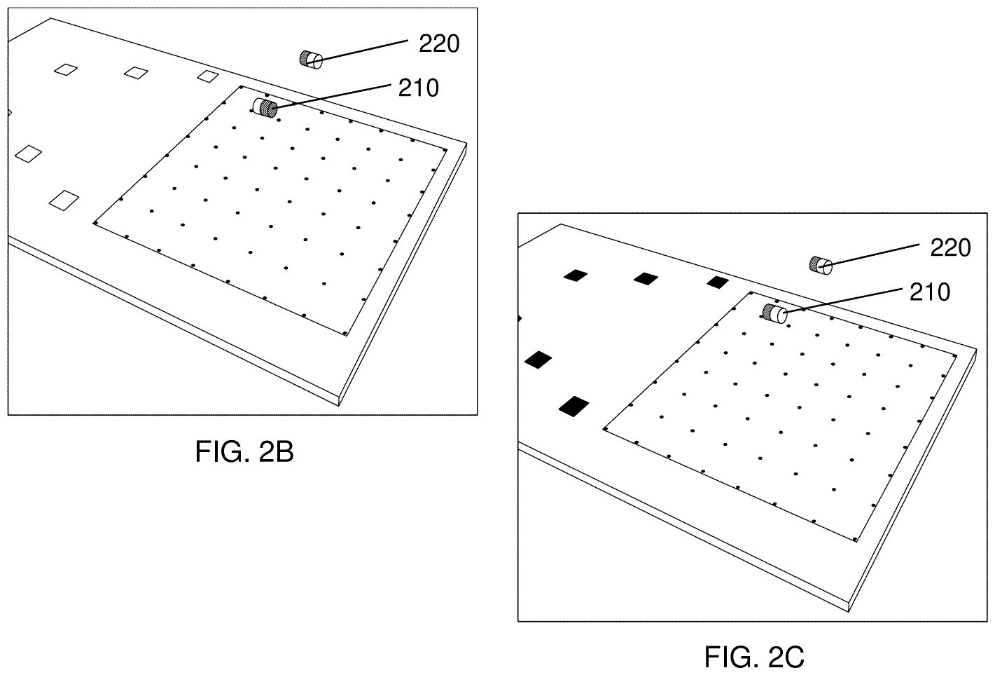

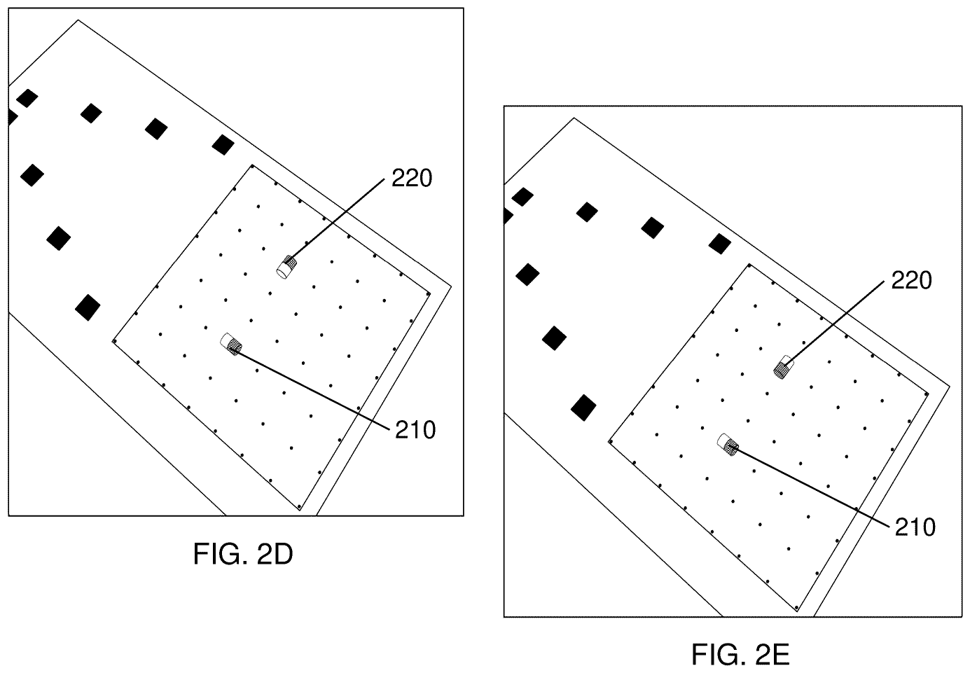

In some embodiments, multiple beacons, which can be configured as a beacons object, are fixed in distance and orientation to each other. In an example, the beacons (210, 220) are linear 20 mm apart (as shown in FIG. 2A). In another example, the beacons are in parallel, 30 mm apart with poles matching (e.g., the south pole of the first magnet (210) is adjacent to the south pole of the second magnet (220), as shown in FIG. 2C). In yet another example, the beacons are arranged with poles opposite each other (e.g., the south pole of the first magnet (210) is adjacent to the north pole of the second magnet (220), as shown in FIG. 2B). In yet another example, the beacons (210, 220) are arranged perpendicularly with respect to their magnetic axes (as shown in FIGS. 2D and 2E). In yet another example, three beacons are arranged in the shape of a triangle, and four beacons are arranged in the shape of a rectangle; more generally, three or more beacons are arranged on a periphery of a convex polygon. Multiple beacons help disambiguate the localization solution when the beacons are in a fixed location and the sensors object is free moving (and is further detailed in Section 5).

Once the configuration of the beacons has been fixed in orientation, polarity, and distance, an 8-parameter model can be used to determine the location of the beacons. The 7 or 8 parameters represent the [X, Y, Z] center of the fixed structure (or a translation thereof), a 4-parameter orientation (axis angle, rotation quaternion) or 3 Euler angles, and a final parameter representative of the magnetic field strength of the beacons. If the strength of the beacons is not uniform, additional parameters may be used. If there are errors expected in the fixed structure, additional parameters for the errors may be used.

In some embodiments, an iterative method (e.g., as discussed in the context of FIG. 1) can be used to solve the 8-parameter problem.

In some embodiments, sets of beacons are configured to use their own pattern. In some examples, three beacons can be removably affixed to the teeth. In other examples, four beacons can be used. In addition, or in the alternative, the multiple beacons can be connected using a flexible PCB structure (e.g., a ribbon cable), which improves localization due to the additional structure available and provides some mechanical strain relief. In these cases, each set of beacons with their own pattern will find their localization solution using either the single, or multiple, beacon localization method. Once complete, these separate solutions can be combined to remove ambiguity and/or improve the accuracy of the solution.

### 2.2.1 Beacons Using a Sine Wave

In some embodiments, a beacon may use a sine wave to generate a varying magnetic field. In this case, the method of determining the magnetic field generated by each beacon requires applying a demodulation method to the magnetometer measurements. In an example, the frequency and phase of the sine wave is known, and the demodulation method is used to recover the magnetic flux vector B. Alternatively, a discrete Fourier transform (DFT) of the frequency bin or bins is used to determine the magnitude and sign of the magnetic flux vector B at each of the magnetometers. Alternatively, the measurements are filtered and demodulated using traditional methods, e.g., using a phase-locked loop or a curve-fitting algorithm. When using a phase-locked loop, a local oscillator generates a sine wave that is compared to the received signal, and the difference in phase between the two is used to adjust the phase of local oscillator so that it matches that of the received signal. Alternatively, the curve-fitting algorithm estimates the frequency and phase of the sinusoid that is closest to the received signal. In other embodiments, once the magnetometer data has been demodulated by frequency and phase, the localization method can be used to determine the position and orientation of the object that the magnetometers are affixed to. The signal to be recovered during typical demodulation in this case is the sine wave that drives a particular beacon. The received signal is also approximately modulated by the mechanical motion of each sensor relative to the beacon. During demodulation, a bandpass filter is applied with filter center aligned with the driven beacon frequency. The resulting bandpassed signal is then multiplied by a sinusoid or by a complex exponential to move the received signal to baseband. This leaves one or more signal images at frequencies outside of baseband that are then removed with a lowpass image selection filter. The bandwidth for the bandpass filter of the demodulation process can be chosen to tradeoff between how much sensor noise is included in the extracted signal, how much motion can be successfully captured by the filter, and how much the retrieved output is delayed relative to the input measurements. The dynamics that can be captured scale directly with the width of the bandpass filter. Likewise, the integrated sensor noise increases with the chosen bandpass filter bandwidth (e.g., since noise energy is linear with filter bandwidth for a white input noise spectrum). Furthermore, the group delay from the filter is inversely proportional to the bandpass filter bandwidth.

In some embodiments, the above-described demodulation methods can be configured to trade-off responsiveness and accuracy. In an example, this is achieved using variable bandwidth by dental handpiece velocity and/or distance from the target. In the case of high velocity movement of the dental tool with mounted sensors, it is desirable to use a higher filter bandwidth to capture the dynamics. Typically, the user would only move the dental tool quickly when not close to the procedure target site on the teeth, i.e., in this case, reduced accuracy can be accepted in exchange for a lower delay and a better ability to capture the higher dynamics. Conversely, when very close to, for example, a drill site, the user will be moving the tool much more slowly. When close to target and moving slowly, it is helpful instead to use a narrower filter bandwidth. In this close and slow case, accuracy is paramount, and the narrower filter bandwidth results in improved accuracy for low speed motion due to the reduction in sensor and environmental noise included in the measurement along with the recovered signal. However, the narrower filter inherently results in a higher delay, which is acceptable in this case due to the much lower tool velocity.

More generally, a higher bandpass filter bandwidth is used when the dental tool is farther away and moving quickly, and a lower bandpass filter bandwidth is used when the dental handpiece is close to target and moving slowly. Bandwidth adjustment decisions can be made in response to exceeding a threshold value of a metric related to tool motion speed and target distance. In an example, this can be a binary decision, which can be implemented with hysteresis to limit fast changes between the bandwidth options. A continuously variable bandwidth could be used such that when very far away and moving very fast, the bandwidth is chosen to be maximum, and for very close and moving very slowly, the bandwidth is set to minimum, and the bandwidth is set to a monotonically changing value when moving from one extreme to the other.

In some embodiments, when using low bandwidth filtering while close to the target site, a higher bandwidth filter can also be employed in parallel with the low bandwidth filter to detect when velocity has rapidly increased. When rapid velocity increase is detected, the filter bandwidth is rapidly increased so that the long delay time from the lower bandwidth operation does not result in interference with patient anatomy due to calculating tool target movements with significant lag (e.g., that are too far behind in time).

In some embodiments, the demodulation method is not used, but the phase of the sine wave is used as another variable in the localization method, which can be solved directly or iteratively.

### 2.2.2 Beacons Using Different Frequencies

In some embodiments, two beacons use different frequencies, and each frequency can be treated independently. In this case, the localization method starts with demodulating the known phase and frequency into a magnitude and sign of the magnetic field from the beacon (as discussed earlier), and then determining the position and orientation of each beacon relative to the sensors object. Once this is known, the relative position of each object to each other can be determined. Then the magnetic field measurements can be combined, and the localization method for multiple beacons can be used.

### 2.2.3 Beacons Using Other Signals

In some embodiments, the beacons are controlled such that their signals can be unambiguously detected by the magnetic sensors. In an example, the beacons are operated simultaneously. In another example, the beacons are operated sequentially. In both examples, each coil in each of the beacons can be unambiguously identified based on its corresponding reference location by the system.

In some embodiments, the beacons may emit spread spectrum signals, with each beacon emitting a unique identifier based on a pseudo-random number (e.g., similar to the operation of GPS). Alternatively, the coils across the multiple beacons may be operated simultaneously at the same frequency, which treats the coils as being spatially distributed. In some examples, the coils are operated during the entire measurement, whereas in other examples, they are operated only some of the time.

In some embodiments, the beacon signals are periodic pulses with fundamental frequencies analogous to the sinusoidal signals, but with more complex pulse shapes which have additional harmonic overtones, e.g., a triangle wave, a sawtooth wave, etc.

### 2.3 Additional Aspects of Localization

In some embodiments, when using the iterative approach to find a localization solution, following the gradient from an initial guess that has significant error may lead a local minimum of the solution that also has significant error. If the absolute minimum (or global minimum) is not found, the localization solution will be wrong.

One approach to ensure that the absolute (or global) minimum is found includes testing multiple initial guesses over the entire range of possible beacon locations. Once tested, the resulting convergence metric values are compared to find the absolute minimum, which would correspond to the correct (global) localization solution. In an example, for the X parameter, the range may be within 100 mm of the sensors, and thus, the range for X would be set to [−100 mm, 100 mm]. Then the spacing could be set based on whether the gradient search was set to operate in a coarse mode (e.g., with a spacing of greater than 25 mm), an intermediate mode (e.g., with a spacing between 25 mm and 5 mm), or a fine mode (e.g., with a spacing of less than 5 mm). In this example, an intermediate mode is assumed with a spacing of 10 mm, which results in 21 initial guesses for the X position. This approach would be similarly followed for all the parameters, e.g., in a five-parameter model, for each of (x, y, z, θ, ϕ). Then the total number of initial guesses will be the product of the number of guesses for each parameter. Herein, if there are 21 initial guesses for each of x, y, and z, 10 initial guesses for θ, and 10 initial guesses for ϕ, there would be 21×21×21×10×10=(21)^{3×}(10)²=926,100 iterations to check. Once completed, the minimum is the localization solution (the global minimum), and all others are local minimums and correspond to incorrect localization solutions. Once the correct localization solution has been found, the number of initial guesses in other scenarios can be reduced to exactly one, i.e., the last localization solution that was determined to be the global minimum.

In some embodiments, the number of initial guesses can be reduced by reducing the range of the parameter, e.g., reducing the range of X from [−100 mm, 100 mm] to [−1 mm, 1 mm]. In other embodiments, a different spacing mode can be used, e.g., using a coarse mode with a spacing of 25 mm results in 9 initial guesses, as compared to 21 initial guesses when using a spacing of 10 mm.

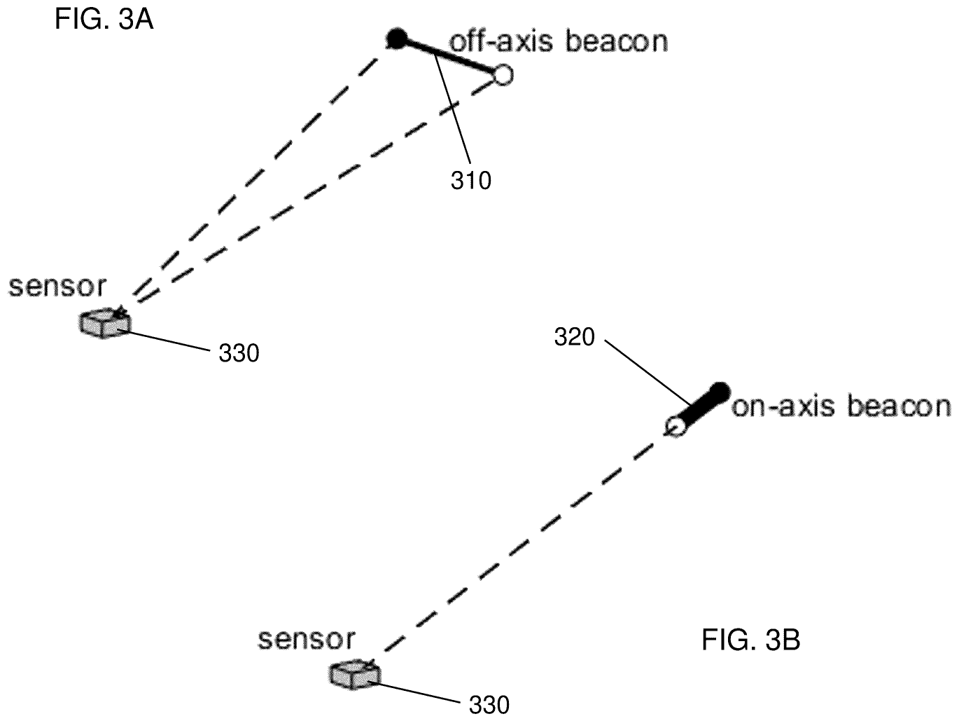

In yet other embodiments, the sensor measurements can be used to reduce the number of initial guesses. Herein, it is firstly noted that when a beacon (310) has its pole equidistant from a sensor (330), it will produce the minimum magnitude for that position. This is referred to as an off-axis beacon, and is illustrated in FIG. 3A. Similarly, when a beacon (320) has its poles form a line with the sensor (330), it will produce the maximum magnitude for that position. This is referred to as the beacon being on-axis, and is illustrated in FIG. 3B. The on-axis and off-axis field strengths can be used to eliminate initial guesses that are impossible or infeasible. In some embodiments, and when the beacon magnetic field strength is known, a minimum and maximum distance to each sensor can be independently determined. Then, the intersection of all these ranges are the only initial guesses that need to be checked.

In some embodiments the beacon magnetic field strength may not be known. In this case, using two or more sensor measurements will produce a bounded region that will reduce the number of initial guesses.

The described embodiments further provide techniques for combining the different beacons. As discussed, a single beacon will ambiguously determine multiple locations for a sensors object. With two or more beacons fixed together (termed the beacons object) that can be uniquely identified, this ambiguity is removed. Because there are errors on each beacon's localization solution, the combination of different beacons will not exactly match the calibration of the beacons object. Various methods for determining the sensors object position from two or more beacons and the beacon calibration are described.

In some embodiments, the localization solutions from the multiple beacons are averaged. To average the orientation, the orientation difference between the beacons object calibration and each beacon's orientation is first determined. The average of each difference is then determined. Next, the beacons and the beacons calibration are rotated into the same orientation using the averaged orientation, and the [x, y, z] offset is averaged to the beacons calibration. This offset and orientation is the averaged sensors object localization solution.

In some embodiments, a weighting factor is used to compute the localization solution. Herein, each beacon is characterized by several parameters that describe its solution, e.g., a convergence metric, a distance to each sensor, a magnetic field strength, a signal to noise ratio (SNR), etc. Using these parameters, a set of weights can be assigned to each beacon that determines how much of that beacon's localization solution is used to determine the sensors object location and orientation. In some examples, beacons with a higher convergence metric are assigned a higher weight, whereas beacons further from the sensors object are assigned a lower weight. In other examples, the weighting factor(s) can be based on a combination of multiple parameters. Once the weighting factors (or weights) have been determined, a weighted average can be used in the same fashion as discussed above in the case of the simple average.

In the described embodiments, when the magnetic sensors are much closer to one beacon than to the others, soft-iron distortion of the received fields due to the high-permeability core material of the nearby beacons can increase position and orientation errors. Approaches to mitigate this issue include (a) imposing geometric constraints to ensure that the sensors do not get extremely close to any single beacon with core material (and which is related to the mechanical design of the dental handpiece or tool), and/or (b) eliminating this effect by using only coils without a high-permeability core.

As discussed earlier, the localization methods may directly decode the signals received from the beacons, or alternatively, the phase and/or strength of the beacon may be added as unknown parameters into the localization method.

### 3 Examples of Registration

Image registration, which is part of the second operation discussed in Section 1, is the process of transforming different sets of data into one coordinate system. The data may be multiple photographs, data from different sensors, times, depths, or viewpoints. Registration helps overcome issues such as image rotation, scale, and skew that are common when overlaying images.

In the described embodiments, a registration device is used to perform the registration process after beacons are mounted to teeth or some other object, and results in the distance and orientation of the beacons to the teeth or other object being precisely determined, i.e., the coordinates of the teeth, as represented by the model in the reference frame of the beacons, is estimated. The registration system uses a scan of the teeth or object, either by laser, raster, camera, or another method, to create a three-dimensional (3D) representation of the teeth or object. The output of the registration system is the translation, rotation, and scaling to be applied to the model so that it fits within the measurements from the registration device. Beacons are then mounted to the teeth or object based on the output.

In some embodiments, the beacons have markers on them that are recognized by the scanning method, and the scan with both beacons and the teeth or other object is performed at the same time. In other embodiments, a registration device is used about and around the teeth or the object. In an example, the registration device is an object (e.g., a probe) about 3 cm in length with a magnetometer at the tip. In another example, the registration device is a dental handpiece with a plurality of sensors (as discussed further in Section 8). When moving the registration device about the teeth, a map of where the registration device can move will be created using the localization methods described in Section 2. The void (or “keep-out area”) where the registration device cannot move correlates with the map of the teeth. In some embodiments, an inertial measurement unit (IMU) is used to determine the orientation of the registration device, whereas in other embodiments, a camera, laser, or other video system is used.

### 3.1 Examples of Registration Using Explicit Fiducial Markers

Fiducial markers are used in a wide range of medical imaging applications. Images of the same subject produced with two different imaging systems may be correlated by placing a fiducial marker in the area imaged by both systems. Herein, a marker which is visible in the images produced by both imaging modalities is used.

Some existing systems for guided dental surgery use 2D patterns as fiducial markers that are rigidly coupled to the teeth. An optical or X-ray scan on the teeth with the visual 2D fiducial pattern is performed, and compared to a previous optical or X-ray scan on just the teeth for registration. Based on this registration output, a camera is used to determine position and orientation relative to the visual fiducial marker, which is used in conjunction with the previously determined registration output to calculate positions and orientations in the reference frame of the teeth. These existing systems additionally use a variety of splints with radio-opaque markers built into the splints. These splints are rigidly mounted on the teeth, and registration is performed based on comparing a scan of just the teeth to a scan of the teeth with the splints and the visual fiducial marker(s).

In some embodiments, the localization methods described in Section 2 (that are part of the first operation discussed in Section 1) can be used with 2D patterns or 2D matrix barcodes for registration (that is part of the second operation discussed in Section 1).

However, depending on the location of the fiducial markers and the 3D model shape, there may be more than one model orientation that fits the measurements (e.g., as shown in the examples discussed in Section 5), and these existing systems are susceptible to significant performance degradation due to ambiguous positioning. For example, a shape with rotational symmetry such as a square has multiple 90-degree rotations that appear the same. While models of shapes such as teeth are not as symmetrical as simple shapes, there can be local sections that appear similar enough to fit the same sets of measurements.

In some embodiments, these drawbacks of the existing systems are mitigated by performing the registration process using the teeth-mounted coils themselves as fiducial markers. This is based on the coils being relatively radio-opaque for X-rays, and high contrast enough to employ other X-ray-type methods, and the coils themselves being simple visual fiducials for any registration procedure.

In other embodiments, and as described in the next subsection, registration using a magnetic probe and landmarks can be employed to overcome the above-discussed drawbacks of existing systems for guided dental surgery.

### 3.2 Examples of Registration Using Landmarks

In some embodiments, features or landmarks relative to beacons can be used to register the beacons through a laser or video system that was used to generate an original scan of the teeth. An example of a feature is an implanted magnet. Examples of landmarks include (a) one or more points of the canine teeth and (b) the lowest point in a molar, filling, crown, denture, and/or bridge.

In an example implementation, object registration is performed by tracing the outside surface of the teeth or object with a probe whose location can be determined accurately. The probe can be a dental instrument (tool or handpiece) with an AC magnet, a DC magnet, and/or another locating signal source attached. In an example, the probe can be designed based on a 3D touch probe used in CNC (computer numerical control) machining systems. As the probe is moved, the system collects samples of its position in a data structure called a “point cloud.” Once the system has collected a sufficient cloud of points, it can make an estimate of the “keep-out area,” which is the volume occupied by the object being probed. This volume is then matched to a 3D model of the teeth or object by finding the translation and orientation that best matches the observed point cloud.

The 3D models that represent objects are stored as collections of points in space, called “vertexes” (or “vertices”), and triangles known as “faces” or “facets” that are composed of three vertices. Each face (or facet) has several geometric properties that include a central point (e.g., a centroid, a circumcenter, an incenter, or an orthocenter), the line that is perpendicular to the face's three vertices (called the “normal”), and the like. Given a point in space, its proximity to a model face is estimated by combining a few values. Most importantly, it must be determined whether the point is in the interior or exterior of the model. Next, the distance is calculated by drawing a line from the point in the direction of the normal until it intersects the plane of the face. If the intersection is within the boundary of the face, the length of the line gives a measurement of the distance to the face. Finally, the distance of the point to the face's vertexes can be used to estimate the distance to points that are near the intersection of multiple faces.

From these (and other) geometric properties, a weighted combination is computed that determines a value called a “score” for that point in space. Low scores correspond to points that are close to the model faces while still being on the exterior. A total score for the point cloud is calculated by combining the scores for points that are close to the surface of the model. Points farther away from the model, which are surrounded by other points, have their weights reduced to limit the influence of extraneous probe movements on the registration process. Other adjustments to the scoring (e.g., by varying the weights by distance, or penalizing areas with many poor scores) can improve performance.

A weighted combination of values, derived from these geometric properties, generates a value called a “score” to the point in the point cloud. In some examples, the score is high for points that are close to a face or a vertex or low if father away, plus a large negative penalty value if the point is inside the model. Under this convention, points near the model have higher scores, and those farther away have lower or negative scores. As previously described, the distance is taken to an edge if the line from the point along the normal intersects the face without its boundary. Alternatively, a function of distance that grows faster than linear, so points farther away are penalized more, can be employed. A total score for the point cloud is calculated by summing the scores for points that are close to the surface of the model. Points farther away from the model that are surrounded by other points have their weights reduced to limit the influence of extraneous probe movements on the registration process. Performance can be further improved by adjusting the total score by penalizing areas that lack a sufficient coverage of points, or those with many poor scores.

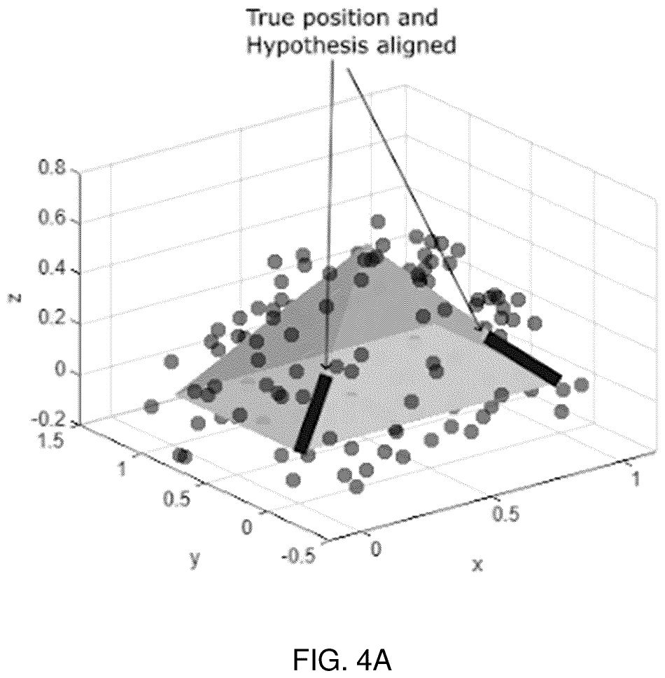

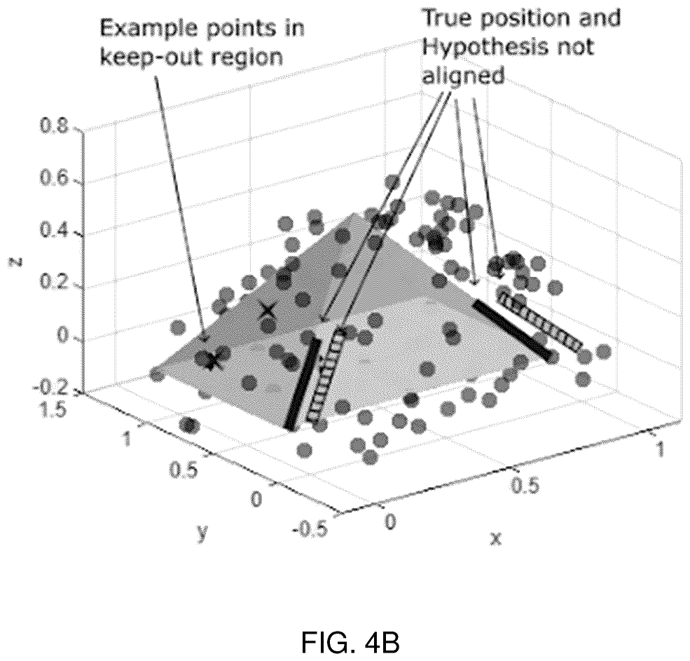

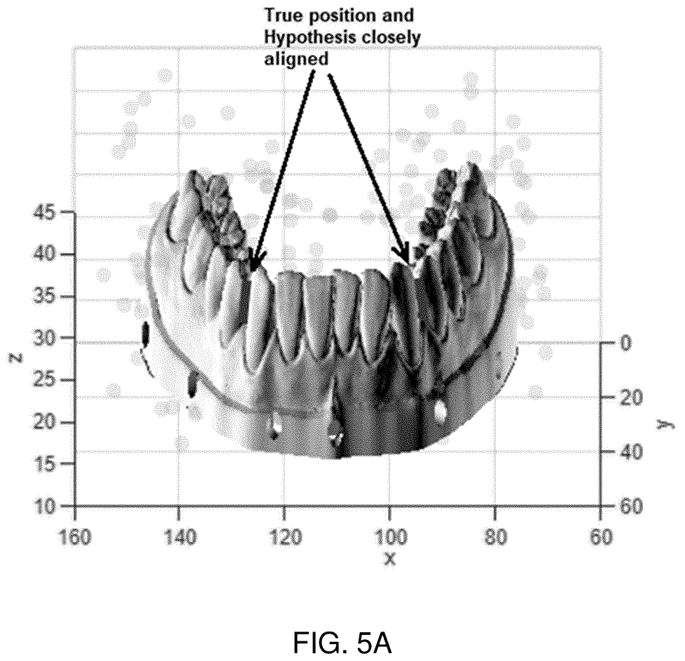

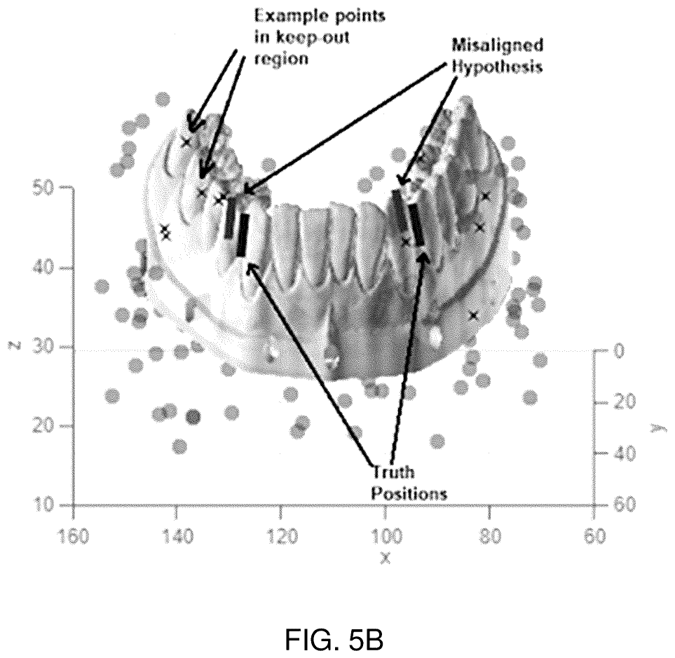

As shown in FIG. 4A, a simplified example of a pyramid whose interior is the keep-out region with a point cloud that matches its translation and rotation. This case has a score of just over 33 with the true and hypothesized positions of the beacons being aligned, and the circular markers signify that these points are on the exterior of the model. In FIG. 4B, the pyramid has been translated by −0.1 along the x-axis. Now, in addition to circular markers, there are some cross (“x”) markers, signifying that they are inside the model, and the score has degraded to −84, with the true positions not being aligned with the hypothesized positions. Examples of scoring with a more accurate model of teeth in the correct location and orientation is shown in FIG. 5A, and the same model is shown misaligned in FIG. 5B. Once the total score is determined, the registration system starts an iterative process to improve the translation and rotation of the model with respect to the point cloud. From the starting point, an incremental translation and rotation is applied to the coordinates (x, y, z, θ, ϕ). Here, (x, y, z) are the 3D cartesian coordinates for translation, θ is a rotation angle about the z axis, and ϕ is an inclination angle from the x-y plane. Small increments are added to the starting coordinates and updated scores are computed for each increment in succession. The change in score for each of the coordinate increments are combined into a vector known as the “gradient”. Following the gradient vector gives the greatest increase in the scoring function; conversely, the negative gradient is the direction of the greatest decrease. In addition to the gradient, there higher-order spatial derivatives that describe properties such as curvature.

In some embodiments, the goal of registration is to find the coordinates that maximize the score of a measured point cloud. Algorithms to maximize the score use the gradient as well as higher-order derivatives. From a suitable starting point, the coordinates are updated with a vector derived from the spatial derivatives and the new score is computed in an increment-and-move process known as refinement. This step is repeated until the change in scoring falls below a threshold known as the convergence value for the algorithm. One simple method known as “gradient descent” moves in the direction of the gradient, scaled by a parameter so it does not overshoot, until the change in score falls below the convergence threshold. Because gradient descent may converge slowly, a related method known as Levenberg-Marquardt improves convergence by adding a quadratic error function as well as the gradient. A third method called BFGS updates an estimate of higher-order derivatives to achieve faster convergence. When the various thresholds and scaling are set appropriately, the algorithm results in a translation and orientation of the model to the point cloud for which the score is a maximum.

### 4 Examples of Calibration

In deployed systems, the angle, distance, and strength of each beacon may vary due to materials and human placement error. Because beacon placement will not be perfect, and there are mounting errors in both position and orientation, the described embodiments provide methods to compensate for errors in placement or mounting. Calibration is part of the third operation discussed in Section 1.

For dental applications, the sensors object needs to be small so as to not get in the way of the dentist or operator, whereas the calibration system can be significantly larger, and contain many more magnetometers to create a highly accurate localization system. Using, for example, the localization methods described in Section 2, a larger sensors object with more sensors can be created, and referred to as the registration object. In some embodiments, the registration object is also the sensors object. In an example, a dental handpiece with sensors therein can be the registration object.

As part of the calibration process, each beacon is driven independently with a signal while the registration object is held stationary relative to the beacons object. This can be done by alternating each beacon, or by driving the beacons with different distinguishable signals. First, this is performed for each beacon independently. Then for pairs of beacons, then for triplets, and so on. Once completed, the multiple beacon locations model will have been determined with high accuracy.

In some embodiments, the registration process (that is part of the second operation discussed in Section 1) can be achieved opportunistically by monitoring the relative motion of the registration object relative to the mounting of the beacons object.

In some embodiments, calibration is not performed, but translation and rotational errors are added as independent variables to the localization system and method (that is part of the first operation discussed in Section 1).

In some embodiments, the registration object can itself be calibrated in a highly controlled environment to account for mounting errors of each sensor. Each sensor can have errors in mounting in both position and rotation.

### 5 Examples of Position Disambiguation

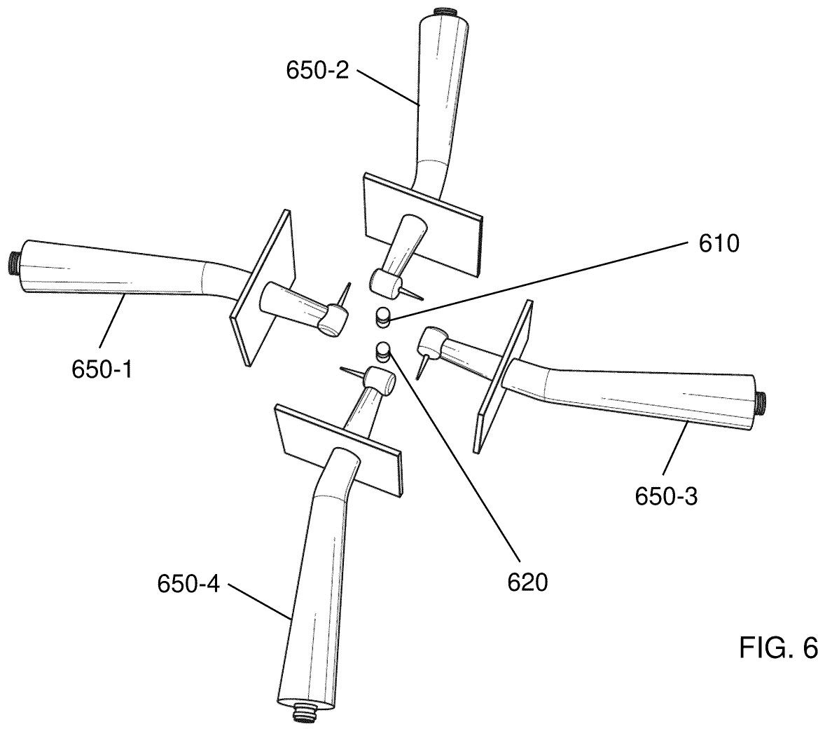

Some of the embodiments described herein can have ambiguous localization solutions due to the symmetry in certain magnet or magnetometer arrangements. In an example, and as shown in FIG. 6, a single cylindrical beacon in a fixed location (or two fixed beacons (610, 620) in a linear configuration) could have the magnetometer object in any location about a circle around the cylinder due to the symmetrical nature of a cylinder (with the candidate locations denoted 650-1, 650-2, 650-3 and 650-4 in FIG. 6). While it can also have the same localization solution underneath the magnetometer object with the poles swapped, the magnetometer objects are oriented and are used only with the beacons above it.

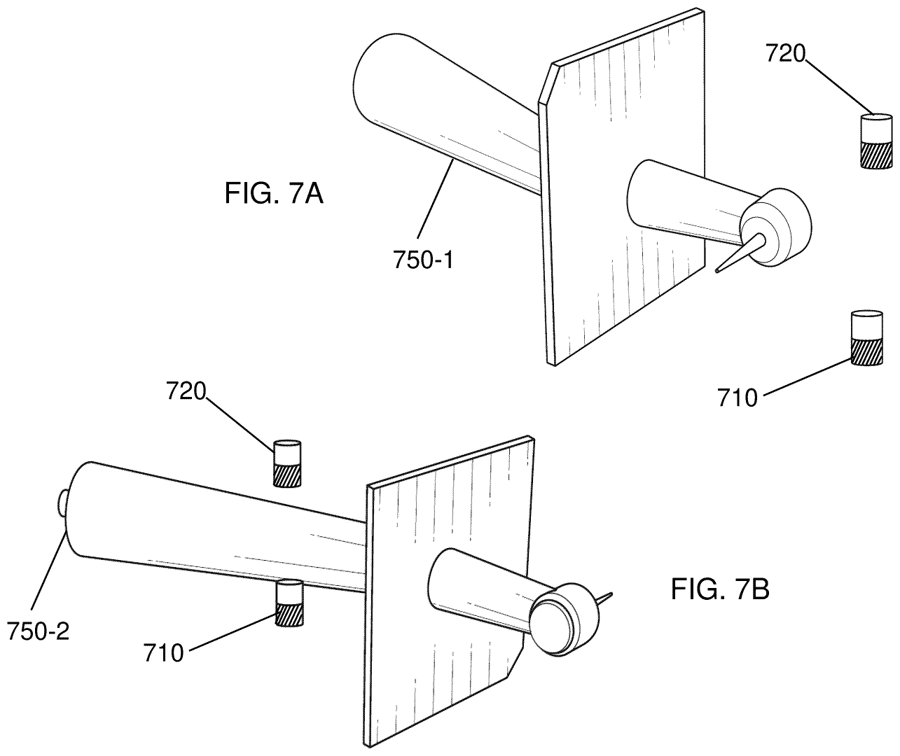

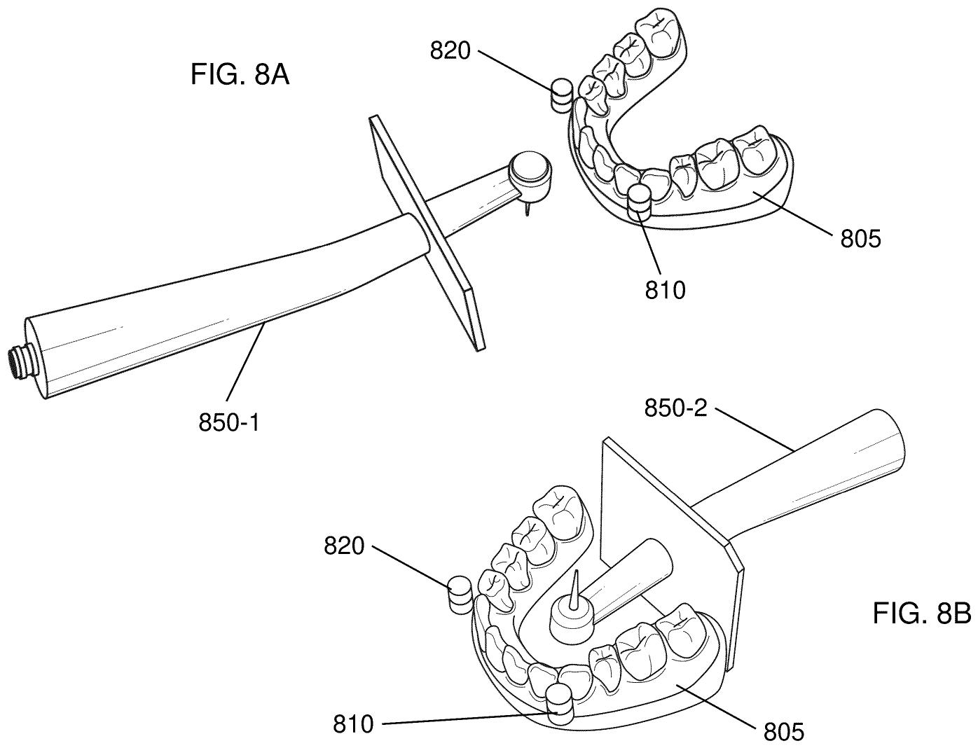

If there are two beacons arranged in a linear configuration, the ambiguity is the same as with a single beacon. Thus, beacon configurations with a mobile magnetometer object are generally chosen to not be linear. If the beacons are in a parallel configuration, there are typically two positions that have the same localization solution. Ambiguous solutions may exist above and below the magnetometer board on the dental handpiece (as shown in FIGS. 7A and 7B, where ambiguous solutions (750-1, 750-2) have the tip of the dental tool pointed in different directions with respect to the beacons (710, 720)), as well as on opposite sides of the beacon object (as shown in FIGS. 8A and 8B, where ambiguous solutions (850-1, 850-2) are on two different sides of the beacons (810, 820) that are affixed to the 3D printed model of the teeth 805).

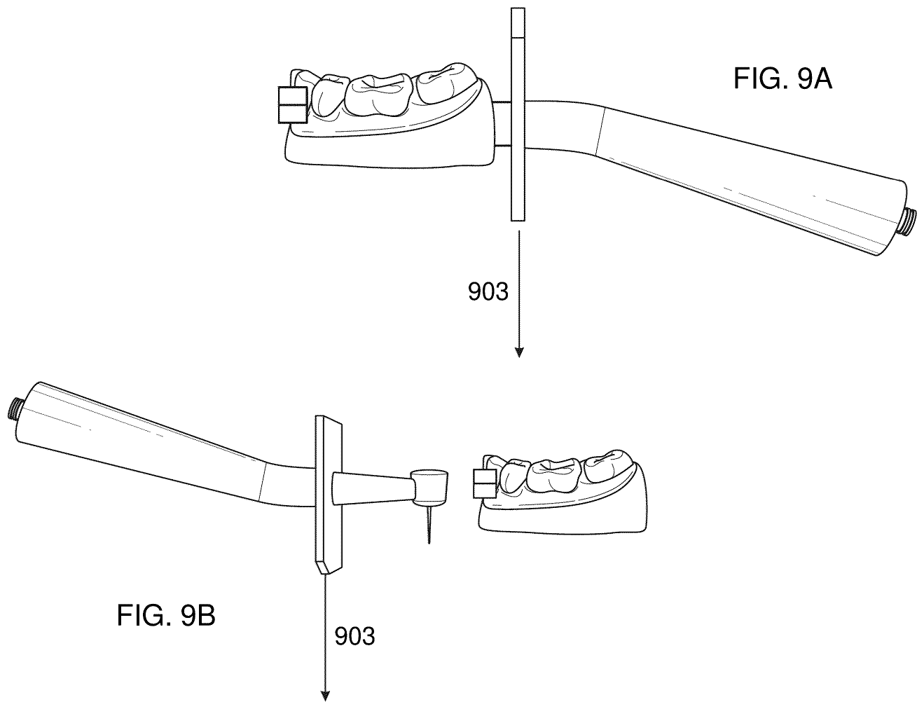

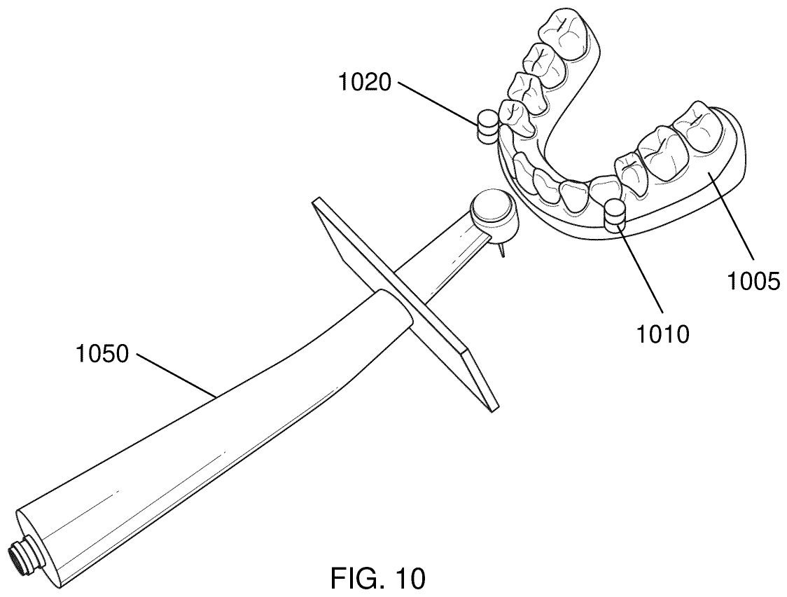

As discussed in the case of FIGS. 8A and 8B, certain beacon object configurations cause the magnetometer object localization orientation to have an inverted solution on the other side of the beacon object. In these cases, the ambiguity of the solution can be resolved by incorporating additional orientation information. For example, the additional information could include:
User input that informs the algorithm and visualization software which solution is the correct one;
As shown in FIGS. 9A and 9B, adding an accelerometer (not explicitly shown therein) to the magnetometer object so that the gravity vector (903) can be determined;
Using of a camera or other imaging system;
As shown in FIG. 10, making each beacon (1010, 1020) in the beacons object operate with a different pattern (e.g., beacon 1010 operating on frequency X and beacon 1020 operating on frequency Y) so that it can be uniquely identified; and/or
Adding additional beacons and fixing them so that there is no symmetry.

### 6 Examples of Digitization in the Described Embodiments

As previously discussed in Section 1, the described embodiments include (a) a first operation that ends with employing the localization method (as described in Section 2) to determine the relative position and/or orientation of each of the sensors relative to the each of the multiple beacons, (b) a second operation that includes a registration process (as described in Section 3) and results in determining the relative position of the dental tool or other object to the teeth or the other object, and (c) a third operation that involves calibration (as described in Section 4) and compensates for imperfections in size, orientation, location, pattern, and strength of the beacons.

In some embodiments, localization methods can be performed using analog X-ray techniques, and the localization outputs can be digitized prior to performing the registration process. In other embodiments, the inputs to the localization methods can be digitized, and all further processing remains in the digital domain.

### 7 Examples of User Feedback

Embodiments of the disclosed technology are used, for example, in guided dental surgery with follows a pre-planned dental procedure path. In some embodiments, the position and orientation outputs of the dental tool from the navigation subsystem are compared with the pre-planned dental procedure path. Feedback relating to a deviation from the planned path, relating to the desired continuation along the path, or both, are provided directly to the tool user, or to a guiding robot, or both. In some examples, no force is provided to continue along the planned procedure path, but resistance to motion is applied so that force in the direction of user's intended movement toward the path or along the path provides much less resistance than force from the user that would diverge from the planned path.

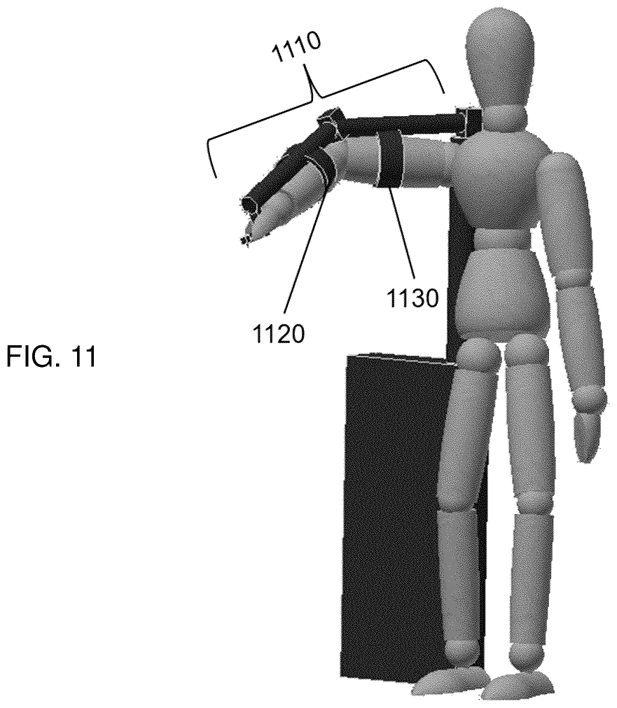

In other examples, the application of dedicated resistance can result in large cost savings to the total system, as it removes the need for a fully-functional guiding robot. Instead, joint resistance to a non-guiding application of force is variable and controlled by the calculated deviation from the dental path. For example, and as shown in FIG. 11, the user can wear the resistive structure over their arm as a jointed sleeve. As shown therein, the jointed sleeve (1110) includes a forearm interface (1120) and an upper arm interface (1130) that interface with the motor and sensing components in the jointed sleeve. In some examples, the calculated deviation from the dental path (e.g., based on the localization, registration and calibration operations described in Sections 2, 3 and 4, respectively) triggers a control circuit that actuates at least one of the motor and/or the sensing components in the jointed sleeve to variably apply force to the user's grip (or hold) of the dental tool.

The described jointed sleeve design advantageously removes the need for a full inverse kinematic model of the jointed structure, relying instead on natural motion of the tool by the user. And as previously noted, it eliminates the need of having a fully-functioning guiding robot for guided dental surgery.

Herein, the amount of resistance for the system to apply is derived from the position and orientation outputs from the navigation subsystem. The relationship between the dental tool and the teeth coordinate system is determined by the position and orientation estimates from the magnetic navigation system, coupled with position and orientation offsets calculated from the registration step, either landmark registration from a magnetic probe (as described in Section 3.2) or from optical or X-ray scans of fiducial markers (as described in Section 3.1).

In some embodiments, other signals to the user can be generated from the same tool-to-teeth relative position and orientation coupled with a surgical plan. These signals can include directional tap, touch, or other haptic feedback such that the position of the haptic signal is in the direction of travel that best matches the surgical plan. Different sequences of haptic feedback can be used for rotational movement and for translational movement. In some examples, a repeated tap at one position on the users hand indicates a direction for translating movement. In other examples, a repeated sweeping tap making an arc over the user's hand indicates an angle change that is needed to match the surgical plan. In yet other examples, these two signaling indicators can be used simultaneously. In yet other examples, other patterns and indicators can be used. In yet other examples, the described signal and haptic feedback can be integrated into the variably-resistive jointed sleeve shown in FIG. 11, which would increase conformance with the surgical plan.

### 8 Example Methods and Implementations of the Disclosed Technology

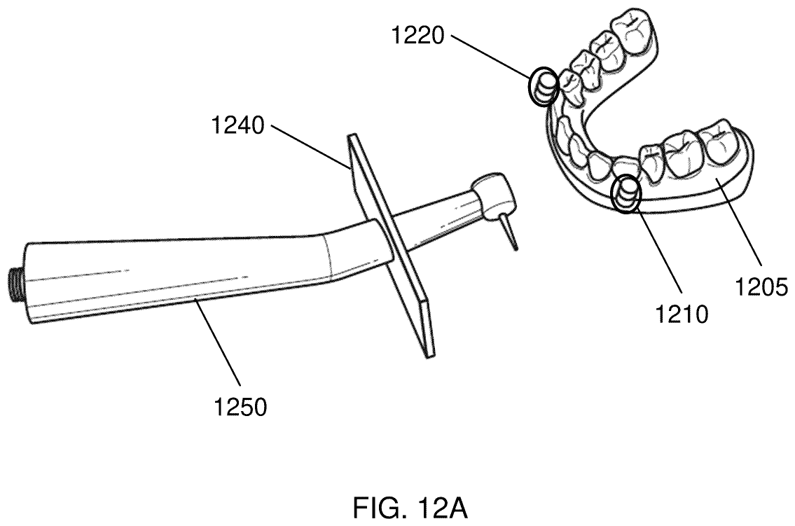

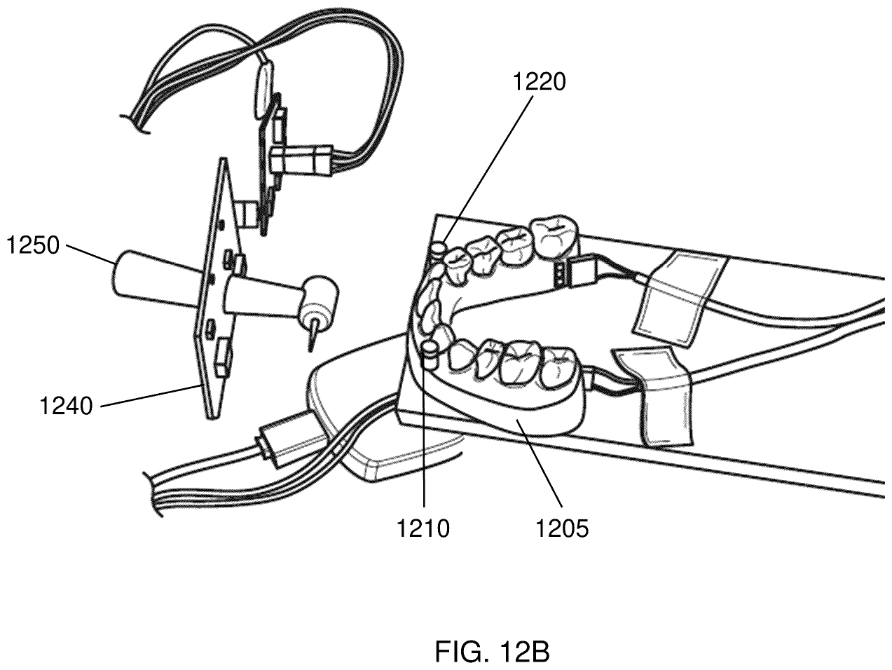

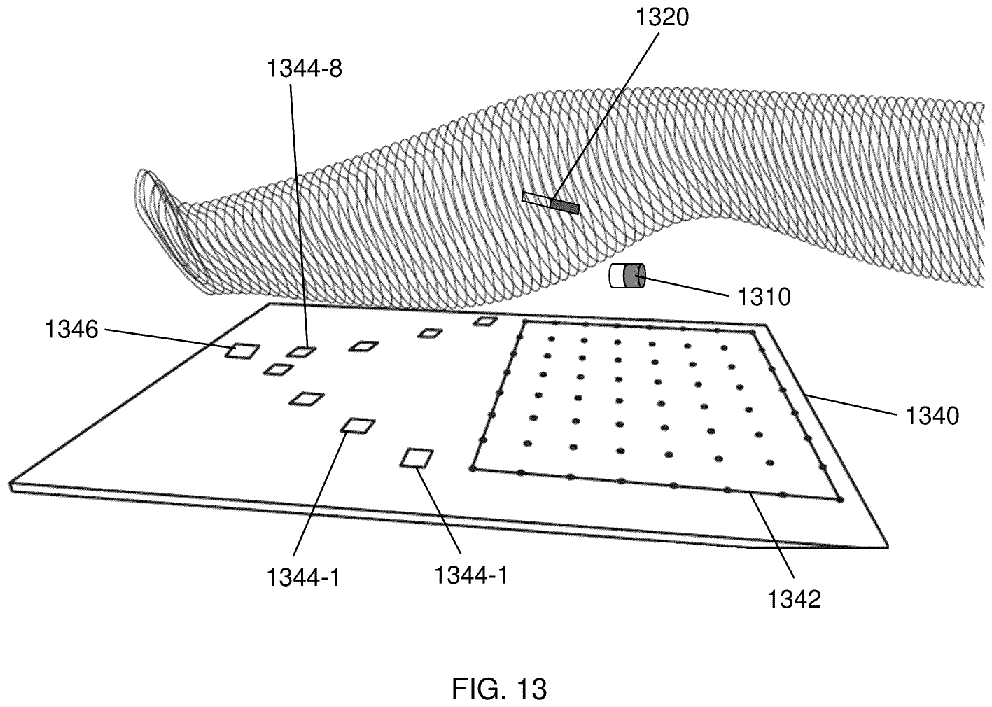

FIG. 12A illustrates a three-dimensional (3D) model of an example positioning system in a visualization software, and FIG. 12B illustrates a prototype of the example positioning system (shown in FIG. 12A). FIG. 13 illustrates a 3D model of another example positioning system in the visualization software.

In some embodiments, an example positioning system, as shown in FIGS. 12A and 12B, includes:
(1) a 3D printed set of plastic teeth (1205) with two AC magnets (1210, 1220) affixed thereon. In some examples, the magnets are arranged parallel to each other at a known separation, and are driven by two different frequencies;
(2) a circuit board (1240) with a dental drill (1250) protruding approximately 20 mm above the circuit board, and further including:
   (2a) sixteen (16) magnetometers arranged in a 4×4 grid with known distances between each pair of magnetometers,
   (2b) one accelerometer to help detect orientation,
   (2c) two (secondary) embedded CPUs to collect the data from the 16 magnetometers, and
   (2d) one (primary) embedded CPU configured to (i) collect the data from the two (secondary) CPUs, (ii) send the data to the PC via a wired USB (or wireless) link, and (iii) drive the AC magnets via a DAC, or receive a phase sync signal from an external source; and
(3) a computing system that includes:
   (3a) a positioning algorithm configured to determine a relative position and location of the 3D printed set of plastic teeth and magnets to the circuit board with the dental drill, and
   (3b) a visualization software displaying the teeth and magnets, as well as the circuit board with dental drill affixed.

In some embodiments, another example positioning system, as shown in FIG. 13, includes:
(1) an AC magnet (1310), autonomously positioned and affixed to a body (the teeth, a leg, an arm, a 3D printed test rig);
(2) a permanent magnet (1320), autonomously positioned and attached to a second body;
(3) a circuit board (1340) including:
   (3a) sixty-four (64) magnetometers (1342) arranged in an 8×8 grid with known distances between each pair of magnetometers,
   (3b) eight (secondary) embedded CPUs (1344-1, . . . , 1344-8) to collect the data from the 64 magnetometers, and
   (3c) one (primary) embedded CPU (1346) configured to (i) collect the data from the eight secondary CPUs (1344-1, . . . , 1344-8), (ii) send the data to the PC via wired USB (or wireless) link, and (iii) drive the AC magnet via a digital-to-analog converter (DAC), or receive a phase synchronization signal from an external source; and
(4) a computing system that includes:
   (4a) a positioning algorithm to determine the relative position and location of at least one of the three objects relative to each other, i.e., the AC magnet and attached object, the permanent magnet and attached object, and the circuit board and any attached objects, and
   (4b) visualization software displaying the AC magnet, the permanent magnet, and the circuit board.

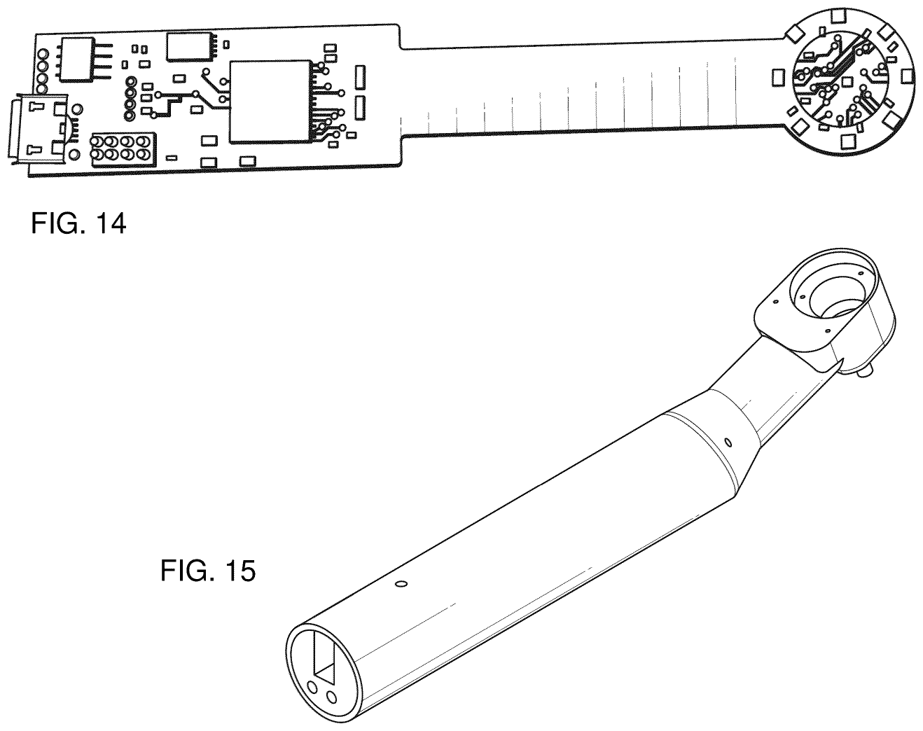

In some embodiments, the dental handpiece contains a printed circuit board (PCB) assembly, as shown in FIG. 14, that serves two main functions:
magnetic field sensing using an array of magnetic field sensors; and
signal generation for electromagnetic coil beacons.

The form factor of the PCB is designed to fit in the dental handpiece head to allow for in-mouth detection of the signal beacons. The flexible PCB construction allows the microcontroller unit (MCU) portion of the assembly to be in the body of the handpiece, while allowing the magnetic sensor array (e.g., magnetometers) to be in the head of the handpiece. The flexible nature of the PCB allows for the angled nature of a ‘contra’ handpiece. Herein, power and communication are supplied over a USB link.

In the examples discussed above, the magnetometers are configured to enter the plane of the patient's mouth. Additionally, or alternatively, the system can include one or more magnetometers that are continuously positioned external to the patient throughout the dental procedure, e.g., on a headrest or side of the surgical bed near the patient's head, etc.

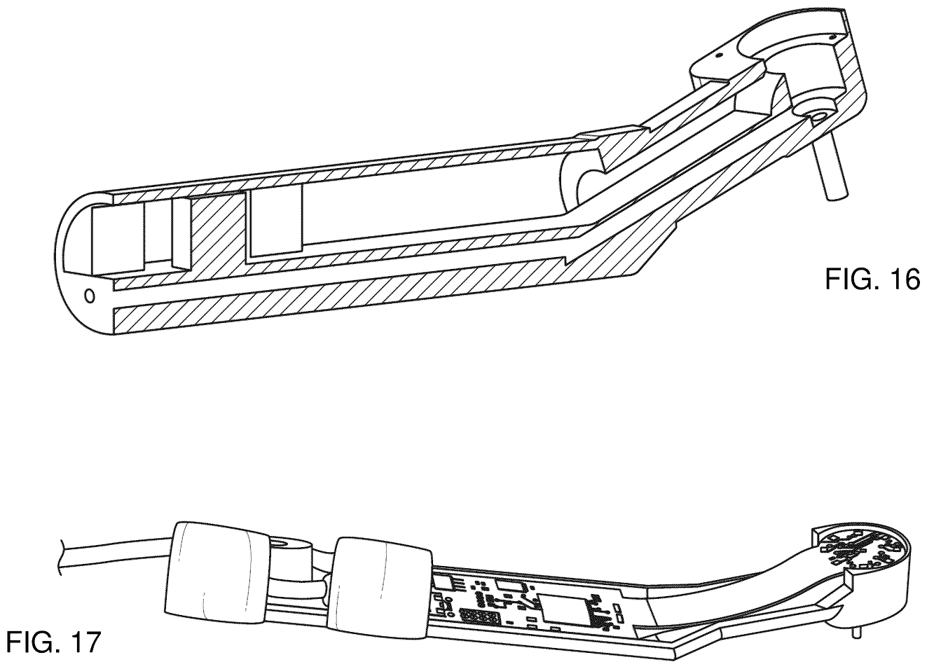

In some embodiments, the dental handpiece (as shown in FIG. 15) includes both a pneumatically-driven turbine and mounting location for the PCB. Detected magnetic signals and electromagnetic coil drive signals generated from the PCB assembly (PCBA) are routed via data and signal cables. Pneumatic feed and exhaust tubes (as shown in FIG. 16) are integrated into the handpiece structure and are routed to the ‘back’ of the handpiece to allow for single connection location with other cable assemblies. The clamshell design allows for placement and secure attachment of PCB within the dental handpiece (as shown in FIG. 17).

In some embodiments, the sensors object is implemented as:
(a) a co-planar N×N grid form, e.g., a 4×4 grid, a 7×7 grid, or an 8×8 grid; or
(b) a co-planar N×N grid form with missing magnetometers. In some examples, a damaged circuit board causes one or more magnetometers to stop working. In other examples, the grid board with missing sensors includes where the drill location is. In yet other examples, the grid board has sensors missing so other components, which include one or more of the following, can be placed there: a processor, an accelerometer, a USB port, an antenna for Bluetooth Low Energy (BLE) or Wi-Fi, an analog out port, a digital input or output connector, and/or a custom component.

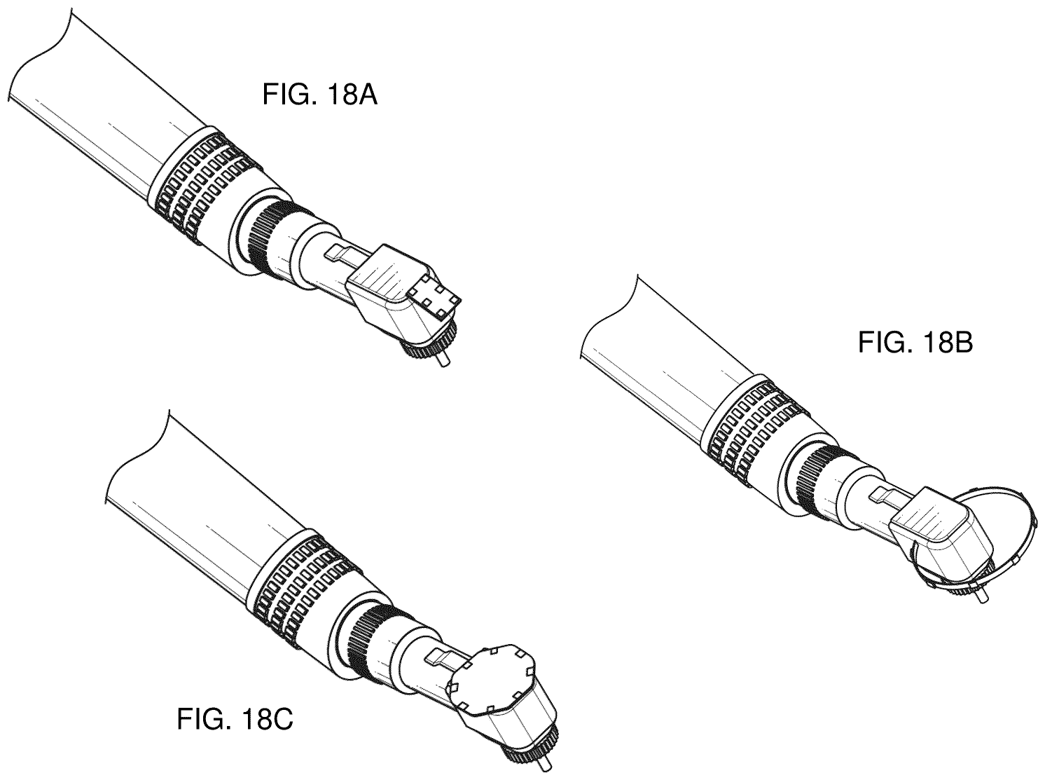

In some embodiments, symmetric and/or non-symmetric placement can be used. In some examples, a planar circuit board includes magnetometers placed in a known fixed configuration (as shown in FIG. 18A). In other examples, magnetometers can be placed non-orthogonally. In yet other examples, magnetometers can be placed on the outside of a ring (as shown in FIG. 18B). In yet other examples, magnetometers can be placed with rotational offsets from each other. In yet other examples, a circular circuit board includes magnetometers placed in a circle around the edge (as shown in FIG. 18C).

In some embodiments, non-planar placement can be used. In some examples, a circuit board made in the form of a hemisphere, and sensors can be placed on the inside or outside of the hemisphere. In other examples, the circuit board is in the form of a half cylinder, with sensors being be placed on the inside or outside. In yet other examples, the circuit board is in the shape of a box or cube, with sensors being placed on the inside or outside. In yet other examples, multiple circuit boards can be rigidly connected in the shape of a hexagon or octagon. In yet other examples, multiple circuit boards can be rigidly connected in any shape.

In some embodiments, the object to which the magnetometers are affixed includes at least one of a dental drill, a dental mirror, a dental x-ray, a head rest, a surgical table, and/or a camera.

In some embodiments, the one or more processors are directly attached to the circuit board, attached via cable to the circuit board, connected via Bluetooth Low-Energy (BLE) or Wi-Fi, or are part of a computing system (e.g., a tablet or a cellphone).

In some embodiments, the data collection rate is (a) typically 60 Hz or above for the visualization tools, (b) collected at lower rates and interpolated, or (c) collected at higher rates and filtered (or down-sampled).

In some embodiments, the data transmission type can use a serial peripheral interface (SPI), an inter-integrated circuit (I2C), USB, Ethernet, universal asynchronous receiver/transmitter (UART), BLE, Wi-Fi, or cellular (e.g., 4G, 5G LTE, and the like).

In some embodiments, the beacons object is implemented as one beacon, or two beacons with a fixed distance between and a fixed orientation to each other. Additionally or alternatively, there can be a hinge or flexible wire between each beacon. In some examples, the size and shape of beacons can be cylindrical, spherical, or oval or “egg” shaped. In other examples, the beacons can be attached to teeth, a dental drill, a dental mirror, a catheter, or a custom medical device. In other examples, the beacon patterns used in the described embodiments can include a single frequency for each beacon, a unique frequency for each beacon, one beacon at a unique frequency and all others at a common frequency, a pseudo-random number pattern, a pulsed on and off signal, or an FM or AM signal. In yet other examples, the beacon can be constructed using a permanent magnet, steel cores of different sizes, shapes, and qualities, a conductive wire of a particular thickness wrapped around the steel core, and variation in the number of turns. Alternatively, the beacon can be constructed without a core made from high-permeability material (e.g., to minimize soft-iron effects that result when a magnetic sensor is much closer to one of multiple beacons).

In some embodiments, the number of beacons (or magnets) that are activated (and used) in one or more of the above-described examples is based on the detected field strength and the distance from the beacons (or magnets). In an example, if the dental handpiece is very close to the beacons, three beacons can be activated, whereas if the dental handpiece is a bit further away, only two beacons on the dental handpiece may be activated. In some embodiments, the strength of the beacons can be dynamically increased or decreased to improve the ability of the sensors to detect that beacon. For example, a beacon strength might be decreased if it surpasses the range of the sensor, whereas its strength might be increased as its distance from the sensors increases.

The described embodiments provide, inter alia, the following technical solutions:

Solution A1. A system including an object that can determine its location and orientation relative to another object by (a) placing a plurality of magnetic field sensors (e.g., magnetometers) on one object (e.g., a sensors object), and (b) placing a plurality of magnets on the other object in any combination of permanent or electric (e.g., a beacons object). Herein, the system further includes a processor and memory including instructions stored thereupon, wherein the instructions, upon execution by the processor, cause the processor to (i) drive any AC magnets with a programmed frequency and phase, (ii) receive magnetic field measurements from the sensors, and (iii) calculate the position and orientation of the magnetometers object relative to the beacons object.

Solution A2. The system of solution A1, wherein the sensors object is attached to a dental drill.

Solution A3. The system of solution A2, wherein the beacons object is the teeth, the jawbone, or the mouth of a person.

Solution A4. The system of solution A3, wherein there is a second beacons object that is an invasive surgical device.

Solution A5. The system of any of solutions A1 to A4, wherein the sensors are embedded inside a dental drill.

Solution A6. The system of any of solutions A1 to A5, wherein the sensors object is a surgical bed.

Solution A7. The system of any of solutions A1 to A6, wherein the beacons are externally driven by a signal generator.

The described embodiments provide, inter alia, the following additional technical solutions:

Solution B1. A system for estimating a position and an orientation of a dental tool, comprising: a plurality of electromagnets removably affixed to an object positioned in a patient's mouth, wherein the object is movable at least based on movements of the patient's mouth; a plurality of magnetometers affixed to the dental tool, wherein the dental tool is non-stationary; and at least one processor configured to: drive the plurality of electromagnets with at least one programmed signal, receive, from the plurality of magnetometers, magnetic field measurements, and determine, based on the magnetic field measurements, the position and the orientation of the dental tool relative to the object.

Solution B2. The system of solution B1, wherein the dental tool is a component in a robotic-assisted dental procedure system, and wherein the position and the orientation is determined to within a predetermined tolerance.

Solution B3. The system of solution B1, wherein each of the plurality of magnetometers is configured to operate within a dynamic range from 0.1 nT to 1 mT and with a noise floor within a range from 0.3 nT/\sqrt{Hz} to 30 nT/\sqrt{Hz}.

Solution B4. The system of solution B3, wherein the noise floor is approximately equal to 3 nT/\sqrt{Hz}.

Solution B5. The system of any of solutions B1 to B4, wherein determining the position results an ambiguity comprising at least two candidate positions for the position of the dental tool, and wherein the at least one processor is configured to: resolve the ambiguity by selecting one of the at least two candidate positions. In some examples, resolving the position ambiguity is discussed in at least Section 5 and FIGS. 6-10.

Solution B6. The system of solution B5, wherein the ambiguity is resolved based on a user input, an accelerometer in the dental tool, an imaging system, each of the plurality of electromagnets being driven with a different programmed signal, or the plurality of electromagnets being removably affixed to the object in an asymmetric arrangement.

Solution B7. The system of any of solutions BI to B6, wherein the at least one processor is configured to: perform a calibration process to determine (a) a position of each of the plurality of electromagnets and (b) a magnetic field strength around each of the plurality of magnetometers. In some examples, the calibration process (that occurs after the registration process) is described in Section 4.

Solution B8. The system of solution B7, wherein the position or the orientation is determined within a tolerance. In some examples, the tolerance is 0.3 mm in 3D position error and 0.5° in angle error.

Solution B9. The system of any of solutions B1 to B8, wherein the at least one processor is configured to: perform, in conjunction with a registration object, a registration process that (a) determines the position for each of the plurality of electromagnets relative to the object, and (b) compensates for placement and mounting errors associated with the plurality of electromagnets or the plurality of magnetometers.

Solution B10. The system of solution B9, wherein the dental tool is the registration object.

Solution B11. The system of solution B9, wherein the registration object comprises a 3D touch probe.

Solution B12. The system of any of solutions B9 to B11, wherein the at least one processor is configured, as part of the registration process, to: generate a 3D model of the object comprising a plurality of vertices and a plurality of faces, wherein each of the plurality of faces includes at least three vertices, a central point, and a normal vector that is perpendicular to a plane formed by the at least three vertices; generate, based on samples collected from the registration object, a point cloud that is representative of a volume occupied by the object; determine a first plurality of scores, wherein each score is representative of a proximity of a corresponding point of the point cloud to the 3D model and is determined based on a line (a) parallel to the normal vector of a particular face, (b) passing through the corresponding point, and (c) intersecting the plane formed by the at least three vertices of the particular face; and determine, based on the first plurality of scores, at least a translation or a rotation of the 3D model of the object. In some examples, the registration process, which is described in Section 3, can use either fiducial markers (see Section 3.1) and/or landmarks (see Section 3.2).

Solution B13. The system of any of solutions B1 to B12, wherein the at least one programmed signal comprises a plurality of spread spectrum signals, and wherein each of the plurality of spread spectrum signals is associated with a distinct pseudo-random number.

Solution B14. The system of any of solutions B1 to B13, wherein the plurality of electromagnets comprises: (a) two bar electromagnets with their magnetic axes arranged in parallel with a south pole of a first electromagnet adjacent to a south pole of a second electromagnet, (b) the two bar electromagnets with their magnetic axes arranged in parallel with the south pole of the first electromagnet adjacent to a north pole of the second electromagnet, (c) the two bar electromagnets with their magnetic axes arranged perpendicularly, or (d) three or more electromagnets arranged on a periphery of a convex polygon.

Solution B15. The system of any of solutions B1 to B14, wherein the plurality of magnetometers are communicatively connected to a flexible printed circuit board.

Solution B16. The system of solution B15, wherein the flexible printed circuit board is designed to fit in a handpiece of the dental tool.

Solution B17. The system of solution B15, wherein the flexible printed circuit board is designed to fit on a handpiece of the dental tool.

Solution B18. The system of any of solutions B15 to B17, wherein the flexible printed circuit board comprises a substrate made from a flexible material.

Solution B19. The system of any of solutions B1 to B18, wherein a strip of flexible material physically connects at least two of the plurality of electromagnets.

Solution B20. The system of solution B18 or B19, wherein the flexible material comprises a polyester resin or a polyimide resin.

Solution B21. The system of any of solutions B1 to B20, comprising: a digital scanning device configured to generate a three-dimensional model of the object, and wherein the at least one processor is configured to: receive the three-dimensional model of the object.

Solution B22. The system of solution B21, wherein the digital scanning device is an intra-oral scanner.

Solution B23. The system of any of solution B1 to B22, wherein at least one electromagnet of the plurality of electromagnets comprises a wire tightly wound around a high-permeability core material.

Solution B24. The system of any of solution B1 to B22, wherein at least one electromagnet of the plurality of electromagnets comprises a tightly-wound wire and excludes a high-permeability core material.

Solution B25. The system of any of solutions B1 to B24, wherein the object comprises one or more teeth, a denture, a 3D printed set of plastic teeth, or a crown.

Solution B26. The system of any of solutions B1 to B25, wherein the dental tool is being used as part of a dental procedure, and wherein the at least one processor is configured to: compare the position or the orientation of the dental tool to a desired position and a desired orientation, respectively, of the dental tool in a predetermined plan for the dental procedure; and provide, based on comparing the position or the orientation and a subsequent step in the predetermined plan for the dental procedure, a feedback to a user of the dental tool that comprises an adjustment to the position or the orientation.

Solution B27. The system of solution B26, wherein the feedback comprises visual or haptic feedback. In some examples, the system of solutions B26 and B27 can be integrated into the variably-resistive jointed sleeve shown in FIG. 11.

Solution B28. The system of any of solution B1 to B27, wherein the at least one processor is configured, as part of receiving the magnetic field measurements, to: receive a set of magnetic field measurements associated with an electromagnet of the plurality of electromagnets that is being driven with a programmed frequency; and filter, using a first bandpass filter, the set of magnetic field measurements, wherein a center frequency of the first bandpass filter is the programmed frequency, and wherein a bandwidth of the first bandpass filter is based on at least one of (a) an instantaneous distance between the dental tool and the object, (b) a target distance between the dental tool and the object, or (c) a velocity of the dental tool. In some examples, the receiving and processing the magnetic field measurements is part of the localization methods, described in Section 2.

Solution B29. The system of solution B28, wherein a group delay associated with the first bandpass filter is configured to be inversely proportional to the bandwidth of the first bandpass filter.

Solution B30. The system of solution B28, wherein the at least one processor is configured to: filter, using a second bandpass filter, the set of magnetic field measurements, wherein a center frequency of the second bandpass filter is the programmed frequency, and wherein a bandwidth of the second bandpass filter is greater than the bandwidth of the first bandpass filter.

Solution B31. The system of solution B30, wherein the at least one processor is configured to: reconfigure the bandwidth of the first bandpass filter based on comparing an output of the first bandpass filter and an output of the second bandpass filter.

Solution B32. The system of any of solutions B1 to B31, wherein the position or the orientation is based on one or more parameters, wherein a parameter of the one or more parameters is associated with a numerical range, and wherein the at least one processor is configured, as part of determining the position and the orientation, to: determine, based on a value of a spacing parameter, a set of initial values that are uniformly spaced within the numerical range for the parameter; determine, for each of the set of initial values, candidate values for the position and the orientation, thereby determining a plurality of candidate values; and select, from the plurality of candidate values, the candidate values for the position and orientation corresponding to a minimum error metric.

Solution B33. The system of solution B32, wherein the at least one processor is configured to reduce a complexity of determining the position and the orientation by: decreasing the value of the spacing parameter, increasing a minimum value of the numerical range, or decreasing a maximum value of the numerical range.

Solution B34. A method for estimating a position or an orientation of a dental tool, comprising: determining a position of each of a plurality of electromagnets removably affixed to an object in a patient's mouth, wherein the object is movable at least based on movements of the patient's mouth, and wherein the position is determined within a tolerance; driving the plurality of electromagnets with at least one programmed signal; receiving, from a plurality of magnetometers removably affixed to the dental tool that is non-stationary, one or more magnetic field measurements when the dental tool is adjacent to the object; and determining, based on the one or more magnetic field measurements, the position or the orientation of the dental tool relative to the object.

Solution B35. The method of solution B34, wherein determining the position and the orientation results in a first ambiguity comprising at least two candidate positions for the position of the dental tool and a second ambiguity comprising at least two candidate orientations for the orientation of the dental tool, and wherein the method comprises: resolving the first ambiguity by selecting one of the at least two candidate positions and the second ambiguity by selecting one of the at least two candidate orientations based on a user input, an accelerometer in the dental tool, an imaging system, each of the plurality of electromagnets being driven with a different programmed signal, or the plurality of electromagnets being removably affixed to the object in an asymmetric arrangement.

Solution B36. The method of solution B34, comprising: receiving, from a digital scanning device, a three-dimensional model of the object, wherein determining the position of each of the plurality of electromagnets is based on the three-dimensional model.

Solution B37. The method of solution B34, wherein each of the plurality of magnetometers is configured to operate within a dynamic range from 0.1 nT to 1 mT and with a noise floor within a range from 0.3 nT/\sqrt{Hz} to 30 nT/\sqrt{Hz}.

Solution B38. The method of solution B37, wherein the noise floor is approximately equal to 3 nT/\sqrt{Hz}.

Solution B39. A dental system, comprising: a hand-held dental tool configured to accommodate cither, but not both, of (a) a plurality of magnetometers or (b) a plurality of electromagnets, wherein an other of the plurality of magnetometers or the plurality of electromagnets not accommodated by the hand-held dental tool are configured to be affixed to an object within a patient's mouth; and one or more processors configured to: drive the plurality of electromagnets with at least one programmed signal, receive, from the plurality of magnetometers, magnetic field measurements, and determine, based on the magnetic field measurements, a position and an orientation of the hand-held dental tool relative to the object, wherein both the plurality of electromagnets and the plurality of magnetometers are moveable at least due to movements of the hand-held dental tool and movements of the patient's mouth, respectively.

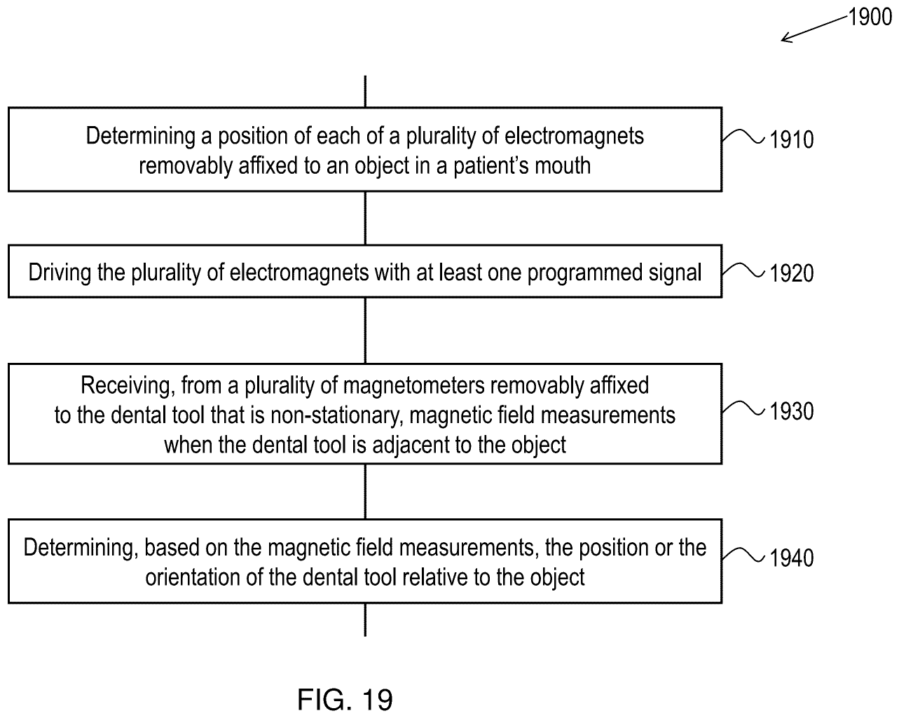

FIG. 19 shows a flowchart of an example method 1900 for estimating a position or an orientation of a dental tool. The method 1900 includes, at operation 1910, determining a position of each electromagnet removably affixed to an object in a patient's mouth.

The method 1900 includes, at operation 1920, driving the plurality of electromagnets with at least one programmed signal. In an example, the at least one programmed signal is a signal that is preprogrammed. In another example, the at least one programmed signal is generated in real-time by a processor (e.g., processor 1902 in FIG. 19). At operation 1930, the method 1900 includes receiving, from a plurality of magnetometers removably affixed to the dental tool that is non-stationary, magnetic field measurements when the dental tool is adjacent to the object.

The method 1900 includes, at operation 1940, determining, based on the magnetic field measurements, the position or the orientation of the dental tool relative to the object.

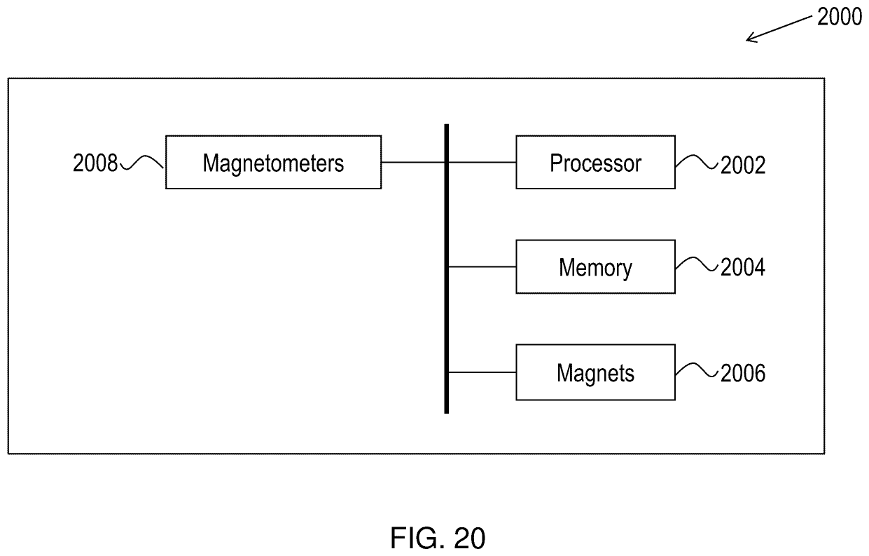

FIG. 20 shows an example of a hardware platform 2000 that can be used to implement some of the techniques described in the present document. For example, the hardware platform 2000 may implement method 1900, or may implement the various modules and algorithms described herein. The hardware platform 2000 may include a processor 2002 that can execute code to implement a method. The hardware platform 2000 may include a memory 2004 that may be used to store processor-executable code and/or store data. The hardware platform 2000 may further include magnets 2006 and magnetometers 2008, which can communicate with the processor 2002 using leads or a wireless protocol. In the described embodiments, magnets 2006 can be electromagnets or permanent magnets. The processor 2002 may be configured to implement localization, registration, and calibration algorithms. In some embodiments, the memory 2004 may comprise multiple memories, some of which are exclusively used by the processor 2002 when implementing the registration and calibration algorithms.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and devices can be implemented as, special purpose logic circuitry, e.g., FPGA (field programmable gate array) or ASIC (application specific integrated circuit).

Processors suitable for the execution of a computer program include, by way of example, both general and special purpose microprocessors, and any one or more processors of any kind of digital computer. Generally, a processor will receive instructions and data from a read-only memory or a random-access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, these are optional. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A system for estimating a position and an orientation of a dental tool, comprising:
a plurality of electromagnets removably affixed to an object positioned in a patient's mouth, wherein the object is movable at least based on movements of the patient's mouth;
a plurality of magnetometers affixed to the dental tool, wherein the dental tool is non-stationary; and
at least one processor configured to:
drive the plurality of electromagnets with at least one programmed signal,
receive, from the plurality of magnetometers, magnetic field measurements, and
determine, based on the magnetic field measurements, the position and the orientation of the dental tool relative to the object.

2. The system of claim 1, wherein the dental tool is a component in a robotic-assisted dental procedure system, and wherein the position and the orientation is determined to within a predetermined tolerance.

3. The system of claim 1, wherein determining the position results an ambiguity comprising at least two candidate positions for the position of the dental tool, and wherein the at least one processor is configured to:
resolve the ambiguity by selecting one of the at least two candidate positions.

4. The system of claim 3, wherein the ambiguity is resolved based on a user input, an accelerometer in the dental tool, an imaging system, each of the plurality of electromagnets being driven with a different programmed signal, or the plurality of electromagnets being removably affixed to the object in an asymmetric arrangement.

5. The system of claim 1, wherein the at least one processor is configured to:
perform a calibration process to determine (a) a position of each of the plurality of electromagnets and (b) a magnetic field strength around each of the plurality of magnetometers, wherein the position is determined within a tolerance.

6. The system of claim 1, wherein the at least one processor is configured to:
perform, in conjunction with a registration object, a registration process that (a) determines the position for each of the plurality of electromagnets relative to the object, and (b) compensates for placement and mounting errors associated with the plurality of electromagnets or the plurality of magnetometers,
wherein the registration object comprises the dental tool or a 3D touch probe.

7. The system of claim 6, wherein the at least one processor is configured, as part of the registration process, to:
generate a 3D model of the object comprising a plurality of vertices and a plurality of faces, wherein each of the plurality of faces includes at least three vertices, a central point, and a normal vector that is perpendicular to a plane formed by the at least three vertices;
generate, based on samples collected from the registration object, a point cloud that is representative of a volume occupied by the object;
determine a first plurality of scores, wherein each score is representative of a proximity of a corresponding point of the point cloud to the 3D model and is determined based on a line (a) parallel to the normal vector of a particular face, (b) passing through the corresponding point, and (c) intersecting the plane formed by the at least three vertices of the particular face; and
determine, based on the first plurality of scores, at least a translation or a rotation of the 3D model of the object.

8. The system of claim 1, wherein the plurality of magnetometers are communicatively connected to a flexible printed circuit board that is designed to fit in a handpiece of a dental tool.

9. The system of claim 1, wherein at least one electromagnet of the plurality of electromagnets comprises a tightly-wound wire.

10. The system of claim 9, wherein the tightly-wound wire is wound around a high-permeability core material.

11. The system of claim 1, wherein the object comprises one or more teeth, a denture, a three-dimensional printed set of plastic teeth, or a crown.

12. The system of claim 1, wherein the dental tool is being used as part of a dental procedure, and wherein the at least one processor is configured to:
compare the position or the orientation of the dental tool to a desired position and a desired orientation, respectively, of the dental tool in a predetermined plan for the dental procedure; and
provide, based on comparing the position or the orientation and a subsequent step in the predetermined plan for the dental procedure, a feedback to a user of the dental tool that comprises an adjustment to the position or the orientation,
wherein the feedback comprises visual or haptic feedback.

13. The system of claim 1, wherein the at least one processor is configured, as part of receiving the magnetic field measurements, to:
receive a set of magnetic field measurements associated with an electromagnet of the plurality of electromagnets that is being driven with a programmed frequency; and
filter, using a first bandpass filter, the set of magnetic field measurements,
wherein a center frequency of the first bandpass filter is the programmed frequency, and wherein a bandwidth of the first bandpass filter is based on at least one of (a) an instantaneous distance between the dental tool and the object, (b) a target distance between the dental tool and the object, or (c) a velocity of the dental tool.

14. The system of claim 13, wherein the at least one processor is configured to:
filter, using a second bandpass filter, the set of magnetic field measurements; and
reconfigure the bandwidth of the first bandpass filter based on comparing an output of the first bandpass filter and an output of the second bandpass filter,
wherein a center frequency of the second bandpass filter is the programmed frequency, and wherein a bandwidth of the second bandpass filter is greater than the bandwidth of the first bandpass filter.

15. The system of claim 1, wherein the position or the orientation is based on one or more parameters, wherein a parameter of the one or more parameters is associated with a numerical range, wherein the at least one processor is configured, as part of determining the position and the orientation, to:
determine, based on a value of a spacing parameter, a set of initial values that are uniformly spaced within the numerical range for the parameter;
determine, for each of the set of initial values, candidate values for the position and the orientation, thereby determining a plurality of candidate values; and
select, from the plurality of candidate values, the candidate values for the position and orientation corresponding to a minimum error metric,
wherein the at least one processor is configured to reduce a complexity of determining the position and the orientation by:
decreasing the value of the spacing parameter;
increasing a minimum value of the numerical range; or
decreasing a maximum value of the numerical range.

16. A method for estimating a position or an orientation of a dental tool, comprising:
determining a position of each of a plurality of electromagnets removably affixed to an object in a patient's mouth, wherein the object is movable at least based on movements of the patient's mouth, and wherein the position is determined within a tolerance;
driving the plurality of electromagnets with at least one programmed signal;
receiving, from a plurality of magnetometers removably affixed to the dental tool that is non-stationary, one or more magnetic field measurements when the dental tool is adjacent to the object; and
determining, based on the one or more magnetic field measurements, the position or the orientation of the dental tool relative to the object.

17. The method of claim 16, wherein determining the position and the orientation results in a first ambiguity comprising at least two candidate positions for the position of the dental tool and a second ambiguity comprising at least two candidate orientations for the orientation of the dental tool, and wherein the method comprises:
resolving the first ambiguity by selecting one of the at least two candidate positions and the second ambiguity by selecting one of the at least two candidate orientations based on a user input, an accelerometer in the dental tool, an imaging system, each of the plurality of electromagnets being driven with a different programmed signal, or the plurality of electromagnets being removably affixed to the object in an asymmetric arrangement.

18. The method of claim 16, comprising:
receiving, from a digital scanning device, a three-dimensional model of the object, wherein determining the position of each of the plurality of electromagnets is based on the three-dimensional model.

19. The method of claim 16, wherein each of the plurality of magnetometers is configured to operate within a dynamic range from 0.1 nT to 1 mT and with a noise floor within a range from 0.3 nT/\sqrt{Hz} to 30 nT/\sqrt{Hz}.

20. A dental system, comprising:
a hand-held dental tool configured to accommodate either, but not both, of (a) a plurality of magnetometers or (b) a plurality of electromagnets,
wherein an other of the plurality of magnetometers or the plurality of electromagnets not accommodated by the hand-held dental tool are configured to be affixed to an object within a patient's mouth; and
one or more processors configured to:
drive the plurality of electromagnets with at least one programmed signal,
receive, from the plurality of magnetometers, magnetic field measurements, and
determine, based on the magnetic field measurements, a position and an orientation of the hand-held dental tool relative to the object, wherein both the plurality of electromagnets and the plurality of magnetometers are moveable at least due to movements of the hand-held dental tool and movements of the patient's mouth, respectively.
